SEQUENCE LISTING (21 sequences)

SEQ ID NO: 1 - DNA (cDNA), 336 bp; double-stranded, unknown
  CDS 1..336
cagtctgtgttgacgcagccgccctcagtctctgcggccccaggacagaaggtcaccatctcctgcactgggagcagctccaacctcggggcaggttatgatgttcactggtaccggcaacttccagggacagcccccaaactcctcatctatgataacaacaatcggccctcaggggtccctgaccgattctctggctccaagtctggcccctcagcctccctggccatctctgggctccaggctgaggatgaggctgattattactgccagtcctatgacagcagcctgaatggttatgtcttcggaactgggacccagctcaccgtcctaggt

SEQ ID NO: 2 - protein, 112 aa
QSVLTQPPSVSAAPGQKVTISCTGSSSNLGAGYDVHWYRQLPGTAPKLLIYDNNNRPSGVPDRFSGSKSGPSASLAISGLQAEDEADYYCQSYDSSLNGYVFGTGTQLTVLG

SEQ ID NO: 3 - DNA (cDNA), 357 bp; double-stranded, unknown
  CDS 1..357
gaggtgcagctggtggagtctgggggaggcttggtacagcctggggggtccctgagactctcctgcgcagcctctggagtctccctcagtggatacaagatgaactgggtccgccaggctccagggaaggggctggaatgggtctcttccattactggtatgagtaattacatacactactcagactcagtgaagggccgattcaccatctccagagacaacgccatgaactcactgtatctgcaaatgaacagcctgacagccgaggacacgggtgtttattattgtgcgacacaaccgggggagctggcgccttttgaccattggggccagggaaccctggtcaccgtctcctca

SEQ ID NO: 4 - protein, 119 aa
EVQLVESGGGLVQPGGSLRLSCAASGVSLSGYKMNWVRQAPGKGLEWVSSITGMSNYIHYSDSVKGRFTISRDNAMNSLYLQMNSLTAEDTGVYYCATQPGELAPFDHWGQGTLVTVSS

SEQ ID NO: 5 - protein, 119 aa; identical to SEQ ID NO: 4

SEQ ID NO: 6 - protein, 98 aa
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCAR

SEQ ID NO: 7 - protein, 138 aa
MGWSCIILFLVATATGVHSEVQLLESGGGLVQPGGSLRLSCAASGVSLSGYKMNWVRQAPGKGLEWVSSITGMSNYIHYSDSVKGRFTISRDNAMNSLYLQMNSLTAEDTGVYYCATQPGELAPFDHWGQGTLVTVSS

SEQ ID NO: 8 - protein, 138 aa
MGWSCIILFLVATATGVHSEVQLVESGGGLVQPGGSLRLSCAASGVSLSGYKMNWVRQAPGKGLEWVSSITGMSNYIHYSDSVKGRFTISRDNAMNSLYLQMNSLTAEDTGVYYCATQPGELAPFDHWGQGTLVTVSS

SEQ ID NO: 9 - protein, 111 aa
QSVLTQPPSVSAAPGQKVTISCTGSSSNLGAGYDVHWYRQLPGTAPKLLIYDNNNRPSGVPDRFSGSKSGPSASLAISGLQAEDEADYYCQSYDSSLNGYVFGTGTQLTVL

SEQ ID NO: 10 - protein, 90 aa
QSVLTQPPSVSGAPGQRVTISCTGSSSNIGAGYDVHWYQQLPGTAPKLLIYGNSNRPSGVPDRFSGSKSGTSASLAITGLQAEDEADYYC

SEQ ID NO: 11 - protein, 128 aa
MGWSCIILFLVATATGVHSELTQPPSVSGAPGQRVTISCTGSSSNIGAGYDVHWYQQLPGTAPKLLIYGNSNRPSGVPDRFSGSKSGTSASLAITGLQAEDEADYYCQSYDSSLNGYVFGTGTQLTVL

SEQ ID NO: 12 - protein, 130 aa
MGWSCIILFLVATATGVHSQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLNGYVFGTGTQLTVL

SEQ ID NO: 13 - DNA (cDNA), 6281 bp; double-stranded, unknown
gacgtcgcggccgctctaggcctccaaaaaagcctcctcactacttctggaatagctcagaggccgaggcggcctcggcctctgcataaataaaaaaaattagtcagccatgcatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcccggggatcgatccgtcgacgtacgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgggtacgtgaaccgtcagatcgcctggagacgccatcgaattctgagcacacaggacctcaccatgggatggagctgtatcatcctcttcttggtagcaacagctacaggtgtccactccgaggtccaactgctcgagtctgggggaggcttggtacagcctggggggtccctgagactctcctgcgcagcctctggagtctccctcagtggatacaagatgaactgggtccgccaggctccagggaaggggctggaatgggtctcttccattactggtatgagtaattacatacactactcagactcagtgaagggccgattcaccatctccagagacaacgccatgaactcactgtatctgcaaatgaacagcctgacagccgaggacacgggtgtttattattgtgcgacacaaccgggggagctggcgccttttgaccattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatagatatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacagcgctggatctcccgatccccagctttgcttctcaatttcttatttgcataatgagaaaaaaaggaaaattaattttaacaccaattcagtagttgattgagcaaatgcgttgccaaaaaggatgctttagagacagtgttctctgcacagataaggacaaacattattcagagggagtacccagagctgagactcctaagccagtgagtggcacagcattctagggagaaatatgcttgtcatcaccgaagcctgattccgtagagccacaccttggtaagggccaatctgctcacacaggatagagagggcaggagccagggcagagcatataaggtgaggtaggatcagttgctcctcacatttgcttctgacatagttgtgttgggagcttggatagcttggacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgatcgagtgtatgactgcggccgcgatcccgtcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 14 - DNA (cDNA), 5679 bp; double-stranded, unknown
gacgtcgcggccgctctaggcctccaaaaaagcctcctcactacttctggaatagctcagaggccgaggcggcctcggcctctgcataaataaaaaaaattagtcagccatgcatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcccggggatcgatccgtcgacgtacgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgggtacgtgaaccgtcagatcgcctggagacgccatcgaattctgagcacacaggacctcaccatgggatggagctgtatcatcctcttcttggtagcaacagctacaggtgtccactccgagctcacgcagccgccctcagtctctgcggccccaggacagaaggtcaccatctcctgcactgggagcagctccaacctcggggcaggttatgatgttcactggtaccggcaacttccagggacagcccccaaactcctcatctatgataacaacaatcggccctcaggggtccctgaccgattctctggctccaagtctggcccctcagcctccctggccatctctgggctccaggctgaggatgaggctgattattactgccagtcctatgacagcagcctgaatggttatgtcttcggaactgggacccagctcaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcaattagcagccccgtcaaggcgggagtggagaccaccacaccctccaaacaaagcaacaacaagtacgcggccagcagctatctgagcctgacgcctgagcagtggaagtcccacagaaggtacagctgccaggtcacgcatgaagggagcaccgtggagaagacagtggcccctacagaatgttcatagttctagatctacgtatgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaaccagctggggctcgacagctcgagctagctttgcttctcaatttcttatttgcataatgagaaaaaaaggaaaattaattttaacaccaattcagtagttgattgagcaaatgcgttgccaaaaaggatgctttagagacagtgttctctgcacagataaggacaaacattattcagagggagtacccagagctgagactcctaagccagtgagtggcacagcattctagggagaaatatgcttgtcatcaccgaagcctgattccgtagagccacaccttggtaagggccaatctgctcacacaggatagagagggcaggagccagggcagagcatataaggtgaggtaggatcagttgctcctcacatttgcttctgacatagttgtgttgggagcttggatcgatccaccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcgcggccgcgatcccgtcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 15 - DNA (cDNA), 1442 bp; double-stranded, unknown
gaattctgagcacacaggacctcaccatgggatggagctgtatcatcctcttcttggtagcaacagctacaggtgtccactccgaggtgcagctggtggagtctgggggaggcttggtacagcctggggggtccctgagactctcctgcgcagcctctggagtctccctcagtggatacaagatgaactgggtccgccaggctccagggaaggggctggaatgggtctcttccattactggtatgagtaattacatacactactcagactcagtgaagggccgattcaccatctccagagacaacgccatgaactcactgtatctgcaaatgaacagcctgacagccgaggacacgggtgtttattattgtgcgacacaaccgggggagctggcgccttttgaccattggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatagatatct

SEQ ID NO: 16 - DNA (cDNA), 762 bp; double-stranded, unknown
gaattctgagcacacaggacctcaccatgggatggagctgtatcatcctcttcttggtagcaacagctacaggtgtccactcccagtctgtgttgacgcagccgccctcagtctctgcggccccaggacagaaggtcaccatctcctgcactgggagcagctccaacctcggggcaggttatgatgttcactggtaccggcaacttccagggacagcccccaaactcctcatctatgataacaacaatcggccctcaggggtccctgaccgattctctggctccaagtctggcccctcagcctccctggccatctctgggctccaggctgaggatgaggctgattattactgccagtcctatgacagcagcctgaatggttatgtcttcggaactgggacccagctcaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcaattagcagccccgtcaaggcgggagtggagaccaccacaccctccaaacaaagcaacaacaagtacgcggccagcagctatctgagcctgacgcctgagcagtggaagtcccacagaaggtacagctgccaggtcacgcatgaagggagcaccgtggagaagacagtggcccctacagaatgttcatagttctagatctacgtatgatcagcct

SEQ ID NO: 17 - protein, 6 aa
EVQLLE

SEQ ID NO: 18 - protein, 6 aa
EVQLVE

SEQ ID NO: 19 - DNA (cDNA), 1899 bp; double-stranded, unknown
  CDS 14..1735
ggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatat

SEQ ID NO: 20 - protein, 574 aa
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 21 - protein, 15 aa
GGGGSGGGGSGGGGS

(19) United States
(12) Patent Application Publication
(10) Pub. No.: US 2005/0175986 A1
(43) Pub. Date: Aug. 11, 2005

(54) Human monoclonal antibody

(75) Inventors: Mitchell S. Gross, Wayne, PA (US); Raymond W. Sweet, Bala Cynwyd, PA (US); Geraldine Taylor, Newbury (GB)

Correspondence Address: GLAXOSMITHKLINE;Corporate Intellectual Property - UW2220, P.O. Box 1539, King of Prussia, PA, 19406-0939, US

(73) Assignees: Smit Kline Beecham Corporation; SmithKline Beecham p.l.c.

(21) Appl. No.: 10/461,576

(22) Filed: Jun. 13, 2003

Related U.S. Application Data
(63) Continuation of application No. 10/163,207, filed on Jun. 5, 2002, now abandoned.
Application No. 10/163,207 is a continuation of application No. 09/567,314, filed on May 9, 2000, now abandoned.

(51) Int. Cl. C12Q 1/70; C07H 21/04; C07K 16/12; C12N 5/06

(52) U.S. Cl. 435/5; 435/69.1; 435/320.1; 435/339; 530/388.3; 536/23.53

(57) ABSTRACT

This invention relates to novel human monoclonal antibodies (mAbs) and to the genes encoding same. More specifically, this invention relates to human monoclonal antibodies specifically reactive with an epitope of the fusion (F) protein of Respiratory Syncytial Virus (RSV). Such antibodies are useful for the therapeutic and/or prophylactic treatment of RSV infection in human patients, particularly infants and young children.

### FIELD OF THE INVENTION

This invention relates to novel human monoclonal antibodies (mAbs) and to the genes encoding same. More specifically, this invention relates to human monoclonal antibodies specifically reactive with an epitope of the fusion (F) protein of Respiratory Syncytial Virus (RSV). Such antibodies are useful for the therapeutic and/or prophylactic treatment of RSV infection in human patients, particularly infants and young children.

### BACKGROUND OF THE INVENTION

Respiratory syncytial virus (RSV) is the major cause of lower respiratory disease in children, giving rise to predictable annual epidemics of bronchiolitis and pneumonia in children worldwide. The virus is highly contagious, and infections can occur at any age. Comprehensive details concerning RSV infection and its clinical features can be obtained from excellent recent reviews by McIntosh, K. and R. M. Chanock, In: “Respiratory Syncytial Virus”, Ch. 38, B. N. Fields ed., Raven Press (1990) and Hall, C. B., In: “Textbook of Pediatric Disease” Feigin and Cherry, eds., W. B. Saunders, pgs 1247-1268 (1987).

RSV is distributed worldwide. One of the most remarkable features of the epidemiology of RSV virus, as mentioned above, is the consistent pattern of infection and disease. Other respiratory viruses cause epidemics at irregular intervals or exhibit a mixed endemic/epidemic pattern, but RSV is the only re spiratory viral pathogen that produces a sizable epidemic every year in large urban centers. In the temperate areas of the world, RSV epidemics have occurred primarily in the late fall, winter or spring but never during the summer. The occurrence and spread of infection within a community is characteristic and easily diagnosed, leading to sharp rises in cases of bronchiolitis and pediatric pneumonia and the number of hospital admissions of young children with acute lower respiratory tract disease. Other respiratory viral agents that occur in outbreaks are rarely present at the same time as RSV.

Primary RSV infection occurs in the very young. Zero to 2 year old infants are the most susceptible and represent the primary affected population. In this group, 1 out of 5 will develop lower respiratory (below larynx) disease upon infection and this ratio stays the same upon reinfection. By 1 year of age, 25-50% of infants have specific antibodies as a result of natural infection and this is close to 100% by age 4-5. Thus, virtually all children have been infected before they have entered school.

Age, sex, socioeconomic and environmental factors can all influence the severity of disease. Hospitalization is required in 1-3% of cases of RSV infection and is usually of long duration (up to 3 weeks). The high morbidity of RSV infection, especially in infancy, has also been implicated in the development of respiratory problems later in life. With current intensive care in the U.S. and the other developed countries, overall mortality for normal subjects is low (less than 2% of hospitalized subjects). However, mortality is much higher in less developed countries and, even in developed countries, mortality is high in certain risk groups such as in infants with underlying cardiac condition (cyanotic congenital heart disease) or respiratory disease (bronchopulmonary dysplasia) where the progression of symptoms may be rapid. For instance, mortality in infants with cyanotic congenital heart disease has been reported to be as high as 37%. In premature infants apneic spells due to RSV infection may occur and, in rare cases, cause neurologic or systemic damage. Severe lower respiratory tract illness (bronchiolitis and pneumonia) is most common in patients under six months of age. Infants who have apparently recovered completely from this illness may display symptomatic respiratory abnormalities for years (recurrent wheezing, decreased pulmonary function, recurrent cough, asthma, and bronchitis).

Immunity to RSV appears to be short-lived, thus reinfections are frequent. The mechanisms by which the immune system protects against RSV infection and reinfection are not well understood. It is clear, however, that immunity is only partially protective since reinfection is common at all ages, and sometimes occurs in infants only weeks after recovery from a primary infection. Both serum and secretory antibodies (IgA) have been detected in response to RSV infection in adults as well as in very young infants. However, the titers of serum antibodies to the viral F or G glycoprotein, as well as of neutralizing antibodies found in infants (1-8 months of age) are 15-25% of those found in older subjects. These reduced titers may contribute to the increased incidence of serious infection in younger children.

Evidence for the role of serum antibodies in protection against RSV virus has emerged from epidemiological as well as animal studies. In adults exposed naturally to the virus, susceptibility correlated well with low serum antibody level. In infants, titers of maternally transmitted antibodies correlate with resistance to serious disease [Glezen, W. P. et al., J. Pediatr. 98:708-715 (1981)]. Other studies show that the incidence and severity of lower respiratory tract involvement is diminished in the presence of high serum antibody [McIntosh, K. et al., J. Infect. Dis. 138:24-32 (1978)] and high titers of passively administered serum neutralizing antibodies have been shown to be protective in a cotton rat model of RSV infection [Prince, G. A. et al., Virus Res. 3:193-206 (1985)].

Children lacking cell-mediated immunity are unable to overcome their infection and shed virus for many months in contrast to children with normal immune systems. Similarly, nude mice infected with RSV virus persistently shed virus. These mice can be cured by adoptive transfer of primed T cells [Cannon, M. J. et al., Immunology 62:133-138 (1987)].

In summary, it appears that both cellular and humoral immunity are involved in protection against infection, reinfection and RSV disease and that although antigenic variation is limited, protective immunity is not complete even after multiple exposures.

RSV, belonging to the family paramyoxoviridae, is a negative-strand unsegmented RNA virus with properties similar to those of the paramyxoviruses. It has, however been placed in a separate genus Pneumovirus, based on morphologic differences and lack of hemagglutinin and neuraminidase activities. RSV is pleomorphic and ranges in size from 150-300 nm in diameter. The virus matures by budding from the outer membrane of a cell and virions appear as membrane-bound particles with short, closely spaced projections or “spikes”. The RNA genome encodes 10 unique viral polypeptides ranging in size from 9.5 kDa to 160 kDa [Huang, Y. T. and G. W. Wertz, J. Virol. 43:150-157 (1982)]. Seven proteins (F, G, N, P, L, M, M2) are present in RSV virions and at least three proteins (F, G, and SH) are expressed on the surface of infected cells. The F protein [SEQ ID NO: 20] has been conclusively identified as the protein responsible for cell fusion since specific antibodies to this protein inhibit syncytia formation in vitro and cells infected with vaccinia virus expressing recombinant F protein form syncytia in the absence of other RSV virus proteins. In contrast, antibodies to the G protein do not block syncytia formation but prevent attachment of the virus to cells.

RSV can be divided into two antigenically distinct subgroups, (A & B) [Mufson, M. A. et al., J. Gen'l. Virol. 66:2111-2124 (1985)]. This antigenic dimorphism is linked primarily to the surface attachment (G) glycoprotein [Johnson, R. A. et al., Proc. Nat'l. Acad. Sci. USA 84:5625-5629 (1987)]. Strains of both group A and B circulate simultaneously, but the proportion of each may vary unpredictably from year to year. An effective therapy must therefore target both subgroups of the virus and this is the reason for the selection of the highly conserved surface fusion (F) protein as target antigen for mAb therapy as will be discussed later.

The induction of neutralizing antibodies to RSV virus appears to be limited to the F and G surface glycoproteins. Of these two proteins, the F protein is the major target for cross-reactive neutralizing antibodies associated with protection against different strains of RSV virus. In addition, experimental vaccination of mice or cotton rats with F protein also results in cross protection. The antigenic relatedness of the F protein across strains and subgroups of the virus is reflected in its high degree of homology at the amino acid level. In contrast, in the two subgroups and various strains of RSV, antigenic dimorphism was linked primarily to the G glycoprotein. The F protein has a predicted molecular weight of 68-70 kDa; a signal peptide at its N-terminus; a membrane anchor domain at its C terminus; and is cleaved proteolytically in the infected cell prior to virion assembly to yield disulfide linked F₂ and F₁. Five neutralizing epitopes have been identified within the F protein sequence [SEQ ID NO: 20] and map to residues 205-225; 259-278; 289-299; 483-488 and 417-438. Studies to determine the frequency of sequence diversion in the F protein [SEQ ID NO: 20] showed that the majority of the neutralizing epitopes were conserved in all of the 23 strains of RSV virus isolated in Australia, Europe, and regions of the U.S. over a period of thirty years. In another study, seroresponses of forty three infants and young children to primary infection with subgroup A or a subgroup B strain showed that responses to homologous and heterologous F antigens were not significantly different, while the G proteins of the subgroup A and B strains were quite unrelated. Moreover, antibody inhibition of virus-mediated cell fusion in vitro versus inhibition of infection correlates best with protection in animal models and fusion inhibition is primarily restricted to F protein specific antibodies.

Prophylactic treatment for RSV infection is thus desirable for the high risk groups of children as well as for all children in underdeveloped countries. However, a vaccine for RSV infection is not currently available. Severe safety issues surrounding an attenuated whole virus vaccine tested in the 1960s, as well as the potential of induced immunopathology associated with the newer candidate subunit vaccines make the prospects of a vaccine in the near future appear remote. To date one drug therapy, Ribavirin, a broad spectrum antiviral, has been approved. Ribavirin has gained only minimal acceptance owing to problems of administration, mild toxicity and questionable efficacy. In the majority of cases, hospitalized children receive no drug therapy and receive only intensive supportive care which is extremely costly. It is clear that there is a need for a safe, effective and easily administered drug for the treatment of RSV infection.

The use of passive antibody therapy in humans is well documented and is being used to treat other infectious diseases such as hepatitis and cytomegalovirus. The feasibility of passive antibody treatment/protection against RSV has been well established using animal models. Most of the earlier passive transfer studies in animals against infectious agents, including RSV, utilized murine mABs. Studies in animals have clearly demonstrated that polyclonal and monoclonal antibody against both F and G glycoprotein can confer passive protection in RSV virus infection when given prophylactically or therapeutically [Prince, et al., supra]. In these studies, passive transfer of neutralizing F or G mAbs to mice, cotton rats or monkeys, significantly reduce or completely prevent replication of the RSV virus in the lungs. However, as discussed above, clearly, the F protein is the more important target for antibody therapy.

Recently, the FDA has approved for use intravenous gammaglobulins (IVIG) isolated from pooled human sera. Initial reports from this study had been encouraging [Groothuis, J. R. et al., pi Antimicrob. Agents Chemo. 35(7):1469-1473 (1991)]. However, generic shortcomings of IVIGs exist and include, without limitation, the fact that such products are human blood derived and grams of antibody often need to be administered to achieve an effective dose.

Alternatively, monoclonal antibodies have been employed. The advantages of such an approach include: a higher concentration of specific antibody can be achieved thereby reducing the amount of globulin required to be given; the reliance on direct blood products can be eliminated; the levels of antibody in the preparation can be more uniformly controlled and the routes of administration can be extended. While passive immunotherapy employing monoclonal antibodies from a heterologous species (e.g., murine) has been suggested (See: PCT Application PCT/US94/08699, Publication No. WO 95/04081), one alternative to reduce the risk of an undesirable immune response on the part of the patient directed against the foreign antibody is to employ “humanized” antibodies. These antibodies are substantially of human origin, with only the Complementarity Determining Regions (CDRs) being of non-human origin. Particularly useful examples of this approach are disclosed in PCT Application PCT/GB91/01554, Publication No. WO 92/04381 and PCT Application PCT/GB93/00725, Publication No. WO93/20210. Clinical trials are on-going to evaluate the efficacy of humanized antibodies for treatment of RSV infection in young children.

A second and more preferred approach is to employ fully human mAbs. Unfortunately, there have been few successes in producing human monoclonal antibodies through classic hybridoma technology. Indeed, acceptable human fusion partners have not been identified and murine myeloma fusion partners do not work well with human cells, yielding unstable and low producing hybridoma lines. However, recent advances in molecular biology and immunology make it now possible to isolate human mABs, particularly directed against foreign infectious agents.

Fully human mAbs to RSV F protein [SEQ ID NO: 20] remain a desirable option for the treatment of this disease. Although some success has been reported in obtaining fragments of such mAbs [Barbas, C. F. et al., Proc. Nat'l. Acad. Sci. USA 89:10164-10168 (1992); Crowe, J. E. et al., Proc. Nat'l. Acad. Sci. USA 91: 1386-1390 (1994) and PCT application number PCT/US93/08786, published as WO94/06448, Mar. 31, 1994)], the achievement of such results is not straightforward. Novel human mABs, when and however obtained, are particularly useful alone or in combination with existing molecules to form immunotherapeutic compositions.

There exists a need in the art for useful prophylactic compositions for the prevention or passive treatment of RSV.

### BRIEF DESCRIPTION OF THE INVENTION

In one aspect, this invention provides fully human monoclonal antibodies and functional fragments thereof specifically reactive with an F protein epitope of RSV and capable of neutralizing RSV infection. These human mABs specific for the F protein of RSV virus may be useful to passively treat or prevent infection.

In another aspect, the present invention provides modifications to neutralizing single chain Fv fragments (scFV) specific for the F protein of RSV produced by random combinatorial cloning of human antibody sequences and isolated from a filamentous phage Fab display library.

In still another aspect, there is provided a reshaped or altered human antibody containing human heavy and light chain constant regions from a first human donor and heavy and light chain variable regions or the CDRs thereof derived from human neutralizing monoclonal antibodies for the F protein of RSV derived from a second human donor.

In yet another aspect, the present invention provides a pharmaceutical composition which contains one (or more) altered or reshaped antibodies and a pharmaceutically acceptable carrier.

In yet another aspect, the invention provides a pharmaceutical composition comprising at least one dose of an immunotherapeutically effective amount of the reshaped, altered or monoclonal antibody of this invention in combination with at least one additional monoclonal, altered or reshaped antibody. A particular embodiment is provided in which the additional antibody is an anti-RSV antibody distinguished from the subject antibody of the invention by virtue of being reactive with a different epitope of the RSV F protein antigen than the subject antibody of the invention.

In a further aspect, the present invention provides a method for passive immunotherapy of RSV disease in a human by administering to said human an effective amount of the pharmaceutical composition of the invention for the prophylactic or therapeutic treatment of RSV infection.

In yet another aspect, the present invention provides methods for, and components useful in, the recombinant production of human and altered antibodies (e.g., engineered antibodies, CDRs, Fab or F(ab)₂ fragments, or analogs thereof) which are derived from human neutralizing monoclonal antibodies (mAbs) for the F protein of RSV. These components include isolated nucleic acid sequences encoding same, recombinant plasmids containing the nucleic acid sequences under the control of selected regulatory sequences which are capable of directing the expression thereof in host cells (preferably mammalian) transfected with the recombinant plasmids. The production method involves culturing a transfected host cell line of the present invention under conditions such that the human or altered antibody is expressed in said cells and isolating the expressed product therefrom.

In still another aspect of the invention is a method to diagnose the presence of RSV in a human which comprises contacting a sample of biological fluid with the human antibodies and altered antibodies and fragments thereof of the instant invention and assaying for the occurrence of binding between said human antibody (or altered antibody, or fragment) and RSV.

Other aspects and advantages of the present invention are described further in the detailed description and the preferred embodiments thereof.

### BRIEF DESCRIPTION OF THE DRAWINGS

FIG. 1A is a graph illustrating the competition of Gλ-1 scFV phage binding with RSV19 mAb [International patent publication No. WO92/04381, published Mar. 19, 1992].

FIG. 1B is a graph illustrating the competition of Gλ-1 scFV phage binding with RSV B4 mAb [International patent publication No. WO93/20210, published Oct. 14, 1993].

FIG. 2 is a graph illustrating virus neutralization by scFV phages, Gλ-1, Gλ-3, and G_{K}-1 with RSV strain 273.

FIG. 3 illustrates the DNA sequence [SEQ ID NO: 1] and protein sequence (amino acids reported in single letter code) [SEQ ID NO: 2] for the Gλ-1 light chain variable region, processed N-terminus through framework IV.

FIG. 4 illustrates the DNA sequence [SEQ ID NO: 3] and protein sequence (amino acids reported in single letter code) [SEQ ID NO: 4] for the Gλ-1 heavy chain variable region, processed N-terminus through framework IV.

FIG. 5 illustrates the cloning strategy used for the construction of the Gλ-1 monoclonal antibody. The heavy chain V region was cloned into the pCD derivative vector as a XhoI-ApaI fragment. The entire light chain V region was cloned into the pCN derivative vector, 43-1pcn, as a SacI-AvrII fragment. Details are described below.

FIG. 6 provides a comparison of the heavy chain amino acid sequences of the Gλ-1 single chain Fᵥ [SEQ ID NO: 5] and various monoclonal antibodies of this invention. The amino acid sequences of the heavy chains for the A [SEQ ID NO: 7] and B [SEQ ID NO: 8] constructs are shown. Numbering of the residues is based on the germline (GL) gene Dp58 [SEQ ID NO: 6], beginning at the mature processed amino terminus and ending at CDR3. The “-” indicates identity to the preceding sequence (eg., A compared to B). Bold residues correspond to the leader region, and to CDRs 1-3.

FIG. 7 provides a comparison of the light chain amino acid sequences of the Gλ-1A single chain Fᵥ [SEQ ID NO: 9] and various monoclonal antibodies of this invention. The amino acid sequences of the light chains for the A [SEQ ID NO: 11] and B [SEQ ID NO: 12] constructs are shown. Numbering of the residues in the Vκ region is based on the germline (GL) gene DpL8 [SEQ ID NO: 10], beginning at the mature processed amino terminus and ending at CDR3. For reference to framework 4, the actual numbering is also shown for Gλ-1A. As in FIG. 6, the “-” indicates identity to the preceding sequence.

FIGS. 8A to 8F illustrate the continuous DNA sequence [SEQ ID NO: 13] of the expression plasmid Gλ-1Apcd containing the RSV neutralizing human Gλ-1 mAb for the heavy chain. The start of translation, leader peptide, amino-terminal processing site, carboxy terminus of the Gλ-1 heavy chain, and Eco RI restriction endonuclease cleavage site are shown.

FIGS. 9A to 9E illustrate the continuous DNA sequence [SEQ ID NO: 14] of the expression plasmid Gλ-1Apcn containing the RSV neutralizing human Gλ-1 mAb for the light chain. The corresponding features for the light chain as for FIGS. 8A-8F are shown.

FIGS. 10A and 10B illustrate the continuous DNA sequence [SEQ ID NO: 15] of the coding region of the heavy chain of plasmid Gλ-1Bpcd. Bolded residues indicate differences from the full vector sequence for Gλ-1Apcd in FIGS. 8A-8F [SEQ ID NO: 13].

FIG. 11 is the DNA sequence [SEQ ID NO: 16] of the coding region for the light chain of plasmid Gλ-1Bpcn. Bolded residues indicate differences from the full vector sequence for Gλ-1Apcn in FIGS. 9A-9E [SEQ ID NO: 14].

### DETAILED DESCRIPTION OF THE INVENTION

This invention provides useful human monoclonal antibodies (and fragments thereof) reactive with the F protein of RSV, isolated nucleic acids encoding same and various means for their recombinant production as well as therapeutic, prophylactic and diagnostic uses of such antibodies and fragments thereof.

I. Definitions

As used in this specification and the claims, the following terms are defined as follows:

“Altered antibody” refers to a protein encoded by an altered immunoglobulin coding region, which may be obtained by expression in a selected host cell. Such altered antibodies are engineered antibodies (e.g., chimeric, humanized, or reshaped or immunologically edited human antibodies) or fragments thereof lacking all or part of an immunoglobulin constant region, e.g., Fv, Fab, or F(ab′)₂ and the like.

“Altered immunoglobulin coding region” refers to a nucleic acid sequence encoding an altered antibody of the invention or a fragment thereof.

“Reshaped human antibody” refers to an altered antibody in which minimally at least one CDR from a first human monoclonal donor antibody is substituted for a CDR in a second human acceptor antibody. Preferrably all six CDRs are replaced. More preferrably an entire antigen combining region (e.g., Fv, Fab or F(ab′)₂) from a first human donor monoclonal antibody is substituted for the corresponding region in a second human acceptor monoclonal antibody. Most preferrably the Fab region from a first human donor is operatively linked to the appropriate constant regions of a second human acceptor antibody to form a full length monoclonal antibody.

“First immunoglobulin partner” refers to a nucleic acid sequence encoding a human framework or human immunoglobulin variable region in which the native (or naturally-occurring) CDR-encoding regions are replaced by the CDR-encoding regions of a donor human antibody. The human variable region can be an immunoglobulin heavy chain, a light chain (or both chains), an analog or functional fragments thereof. Such CDR regions, located within the variable region of antibodies (immunoglobulins) can be determined by known methods in the art. For example, Kabat et al. (Sequences of Proteins of Immunological Interest, 4th Ed., U.S. Department of Health and Human Services, National Institutes of Health (1987)) disclose rules for locating CDRs. In addition, computer programs are known which are useful for identifying CDR regions/structures.

“Second fusion partner” refers to another nucleotide sequence encoding a protein or peptide to which the first immunoglobulin partner is fused in frame or by means of an optional conventional linker sequence (i.e., operatively linked). Preferably the fusion partner is an immunoglobulin gene and when so, it is referred to as a “second immunoglobulin partner”. The second immunoglobulin partner may include a nucleic acid sequence encoding the entire constant region for the same (i.e., homologous—the first and second altered antibodies are derived from the same source) or an additional (i.e., heterologous) antibody of interest. It may be an immunoglobulin heavy chain or light chain (or both chains as part of a single polypeptide). The second immunoglobulin partner is not limited to a particular immunoglobulin class or isotype. In addition, the second immunoglobulin partner may comprise part of an immunoglobulin constant region, such as found in a Fab, or F(ab)₂ (i.e., a discrete part of an appropriate human constant region or framework region) A second fusion partner may also comprise a sequence encoding an integral membrane protein exposed on the S outer surface of a host cell, e.g., as part of a phage display library, or a sequence encoding a protein for analytical or diagnostic detection, e.g., horseradish peroxidase (HRP), β-galactosidase, etc.

The terms Fv, Fc, Fd, Fab, or F(ab′)₂ are used with their standard meanings [see, e.g., Harlow et al., Antibodies A Laboratory Manual, Cold Spring Harbor Laboratory, (1988)].

As used herein, an “engineered antibody” describes a type of altered antibody, i.e., a full-length synthetic antibody (e.g., a chimeric, humanized, reshaped or immunologically edited human antibody as opposed to an antibody fragment) in which a portion of the light and/or heavy chain variable domains of a selected acceptor antibody are replaced by analogous parts from one or more donor antibodies which have specificity for the selected epitope. For example, such molecules may include antibodies characterized by a humanized heavy chain associated with an unmodified light chain (or chimeric light chain), or vice versa. Engineered antibodies may also be characterized by alteration of the nucleic acid sequences encoding the acceptor antibody light and/or heavy variable domain framework regions in order to retain donor antibody binding specificity. These antibodies can comprise replacement of one or more CDRs (preferably all) from the acceptor antibody with CDRs from a donor antibody described herein.

A “chimeric antibody” refers to a type of engineered antibody which contains naturally-occurring variable region (light chain and heavy chains) derived from a donor antibody in association with light and heavy chain constant regions derived from an acceptor antibody from a heterologous species.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity [see, e.g., Queen et al., Proc. Nat'l. Acad. Sci. USA, 86:10029-10032 (1989), Hodgson et al., Bio/Technology, 9:421 (1991)].

An “immunologically edited antibody” refers to a type of engineered antibody in which changes are made in donor and/or acceptor sequences to edit regions in respect of cloning,artifacts, germ line enhancements, etc. aimed at reducing the likelihood of an immunological response to the antibody on the part of a patient being treated with the edited antibody.

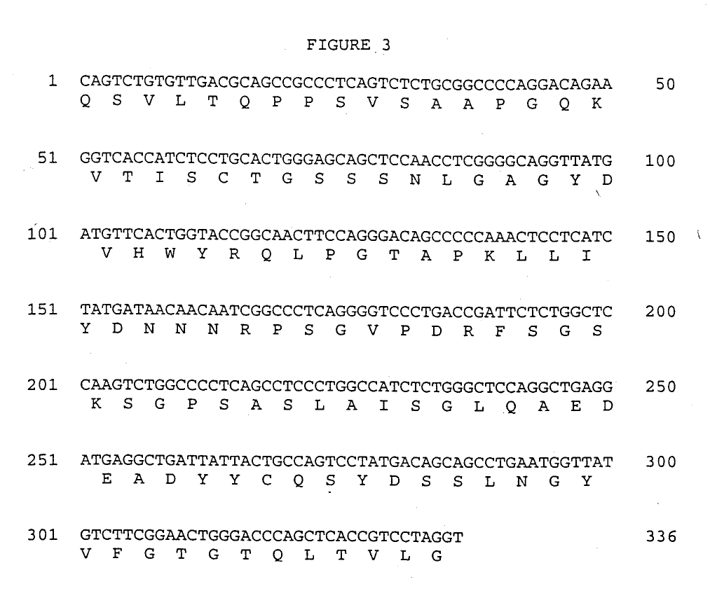

The term “donor antibody” refers to an antibody (monoclonal, or recombinant) which contributes the nucleic acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody. One donor antibody suitable for use in this invention is a Fab fragment of a human neutralizing monoclonal antibody designated as Fab Gλ-1. Fab Gλ-1 is defined as a having the variable light and heavy chain DNA and amino acid sequences Gλ-1 as shown in FIGS. 3, 4, 8A-8F and 9A-9E [SEQ ID NOS: 1-4, 13 and 14].

The term “acceptor antibody” refers to an antibody (monoclonal or recombinant) from a source genetically unrelated to the donor antibody, which contributes all (or any portion, but preferably all) of the nucleic acid sequences encoding its heavy and/or light chain framework regions and/or its heavy and/or light chain constant regions to the first immunoglobulin partner. Preferably a human antibody is the acceptor antibody.

“CDRs” are defined as the complementarity determining region amino acid sequences of an antibody which are the hypervariable regions of immunoglobulin heavy and light chains [see, e.g., Kabat et al., Sequences of Proteins of Immunological Interest, 4th Ed., U.S. Department of Health and Human Services, National Institutes of Health (1987)]. There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

By “sharing the antigen binding specificity or neutralizing ability” is meant, for example, that although Fab Gλ-1 may be characterized by a certain level of antigen affinity, a CDR encoded by a nucleic acid sequence of Fab Gλ-1 in an appropriate structural environment may have a lower, or higher affinity. It is expected that CDRs of Fab Gλ-1 in such environments will nevertheless recognize the same epitope(s) as does the intact Fab Gλ-1. A “functional fragment” is a partial heavy or light chain variable sequence (e.g., minor deletions at the amino or carboxy terminus of the immunoglobulin variable region) which retains the same antigen binding specificity and/or neutralizing ability as the antibody from which the fragment was derived.

An “analog” is an amino acid sequence modified by at least one amino acid, wherein said modification can be a chemical modification, or a substitution or a rearrangement of a few amino acids (i.e., no more than 10), which modification permits the amino acid sequence to retain the biological characteristics, e.g., antigen specificity and high affinity, of the unmodified sequence. For example, (silent) mutations can be constructed, via substitutions, when certain endonuclease restriction sites are created within or surrounding CDR-encoding regions.

Analogs may also arise as allelic variations. An “allelic variation or modification” is an alteration in the nucleic acid sequence encoding the amino acid or peptide sequences of the invention. Such variations or modifications may be due to degeneracy in the genetic code or may be deliberately engineered to provide desired characteristics. These variations or modifications may or may not result in alterations in any encoded amino acid sequence.

The term “effector agents” refers to non-protein carrier molecules to which the altered antibodies, and/or natural or synthetic light or heavy chains of the donor antibody or other fragments of the donor antibody may be associated by conventional means. Such non-protein carriers can include conventional carriers used in the diagnostic field, e.g., polystyrene or other plastic beads, polysaccharides, e.g., as used in the BIAcore (Pharmacia) system, or other non-protein substances useful in the medical field and safe for administration to humans and animals. Other effector agents may include a macrocycle, for chelating a heavy metal atom, or radioisotopes. Such effector agents may also be useful to increase the half-life of the altered antibodies, e.g., polyethylene glycol.

II. Combinatorial Cloning

As mentioned above, a number of problems have hampered the direct application of the hybridoma technology [G. Kohler and C. Milstein, Nature, 256: 495-497 (1975)] to the generation and isolation of human monoclonal antibodies. Among these are a lack of suitable fusion partner myeloma cell lines used to form hybridoma cell lines as well as the poor stability of such hybridomas even when formed. These shortcomings are further exacerbated in the case of RSV because of the paucity of viral specific B cells in the peripheral circulation. Therefore, the molecular biological approach of combinatorial cloning is preferred.

Combinatorial cloning is disclosed generally in PCT Publication No. WO90/14430. Simply stated, the goal of combinatorial cloning is to transfer to a population of bacterial cells the immunological genetic capacity of a human cell, tissue or organ. It is preferred to employ cells, tissues or organs which are immunocompetent. Particularly useful sources include, without limitation, spleen, thymus, lymph nodes, bone marrow, tonsil and peripheral blood lymphocytes. The cells may be optionally RSV stimulated in vitro, or selected from donors which are known to have produced an immune response or donors who are HIV⁺ but asymptomatic.

The genetic information isolated from the donor cells can be in the form of DNA or RNA and is conveniently amplified by Polymerase Chain Reaction (PCR) or similar techniques. When isolated as RNA the genetic information is preferably converted into cDNA by reverse transcription prior to amplification. The amplification can be generalized or more specifically tailored. For example, by a careful selection of PCR primer sequences, selective amplification of immunoglobulin genes or subsets within that class of genes can be achieved.

Once the component gene sequences are obtained, in this case the genes encoding the variable regions of the various heavy and light antibody chains, the light and heavy chain genes are associated in random combinations to form a random combinatorial library. Various recombinant DNA vector systems have been described to facilitate combinatorial cloning [see: PCT Publication No. WO90/14430 supra; Scott and Smith, Science 249:386-406 (1990); or U.S. Pat. No. 5,223,409]. Having generated the combinatorial library, the products can, after expression, be conveniently screened by biopanning with RSV F protein or, if necessary, by epitope blocked biopanning as described in more detail below.

As described herein, it is preferred to use single chain antibodies for combinatorial cloning and screening and then to convert them to full length mAbs after selection of the desired candidate molecules. However, Fab fragments of mAbs can also be used for cloning and screening.

III. Antibody Fragments

The present invention contemplates the use of scFv, Fab, or F(ab′)₂ fragments to derived full-length mAbs directed against the F protein of RSV. Although these fragments may be independently useful as protective and therapeutic agents in vivo against RSV-mediated conditions or in vitro as part of an RSV diagnostic, they are employed herein as a component of a reshaped human antibody. A scFv fragment contains the light and heavy chain variable regions joined by a linker of about 12 amino acids in either a light-linker-heavy or a heavy-linker-light orientation. A Fab fragment contains the entire light chain and amino terminal portion of the heavy chain; and a F(ab′)₂ fragment is the fragment formed by two Fab fragments bound by additional disulfide bonds. RSV binding monoclonal antibodies provide sources of scFv or Fab fragments which can be obtained from a combinatorial phage library [see, e.g., Winter et al., Ann. Rev. Immunol., 12:433-455 (1994) or Barbas et al., Proc. Nat'l. Acad. Sci. (USA) 89, 10164-10168 (1992), which are both hereby incorporated by reference in their entireties].

IV. Anti-RSV Antibody Amino Acid and Nucleotide Sequences of Interest

The Fab Gλ-1 or other antibodies described herein may contribute sequences, such as variable heavy and/or light chain peptide sequences, framework sequences, CDR sequences, functional fragments, and analogs thereof, and the nucleic acid sequences encoding them, useful in designing and obtaining various altered antibodies which are characterized by the antigen binding specificity of the donor antibody.

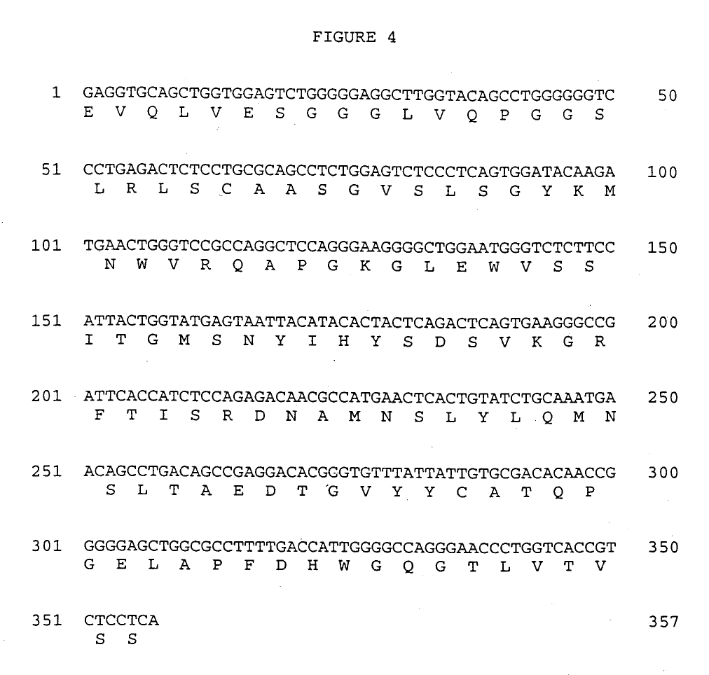

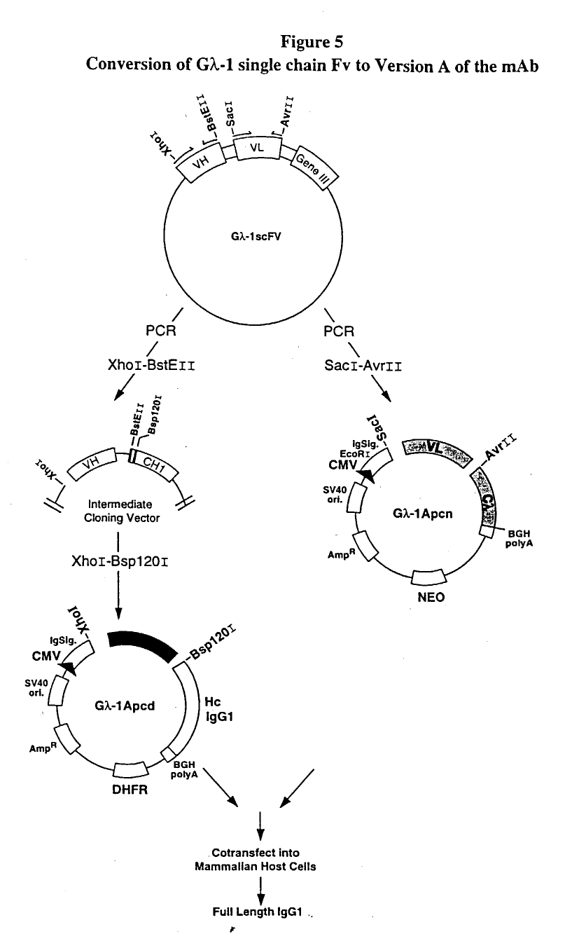

As one example, the present invention thus provides variable light chain and variable heavy chain sequences from the RSV human Fab Gλ-1A and sequences derived therefrom. The heavy chain variable region of Fab Gλ-1A is illustrated by FIGS. 4, 8A-8F and 10A-10B [SEQ ID NOS: 3-4, 13 and 15].

The nucleic acid sequences of this invention, or fragments thereof, encoding the variable light chain and heavy chain peptide sequences are also useful for mutagenic introduction of specific changes within the nucleic acid sequences encoding the CDRs or framework regions, and for incorporation of the resulting modified or fusion nucleic acid sequence into a plasmid for expression. For example, silent substitutions in the nucleotide sequence of the framework and CDR-encoding regions can be used to create restriction enzyme sites which would facilitate insertion of mutagenized CDR (and/or framework) regions. These CDR-encoding regions may be used in the construction of reshaped human antibodies of this invention.

Taking into account the degeneracy of the genetic code, various coding sequences may be constructed which encode the variable heavy and light chain amino acid sequences, and CDR sequences of the invention as well as functional fragments and analogs thereof which share the antigen specificity of the donor antibody. The isolated nucleic acid sequences of this invention, or fragments thereof, encoding the variable chain peptide sequences or CDRs can be used to produce altered antibodies, e.g., chimeric or humanized antibodies, or other engineered antibodies of this invention when operatively combined with a second immunoglobulin partner.

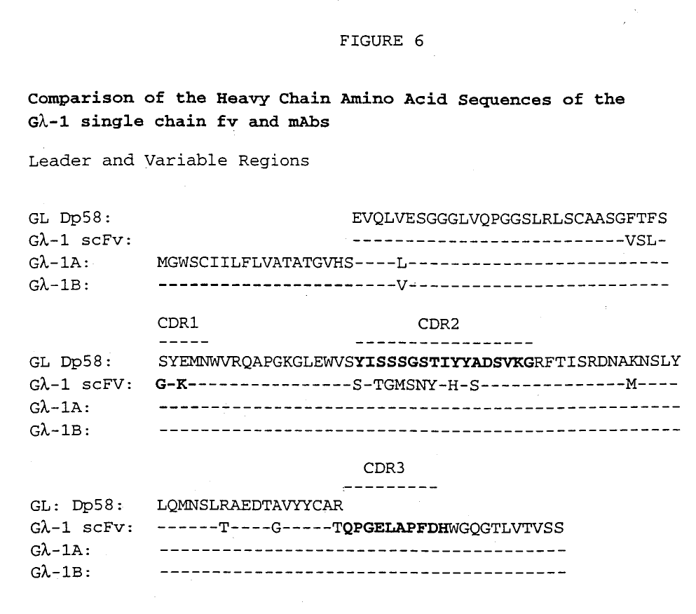

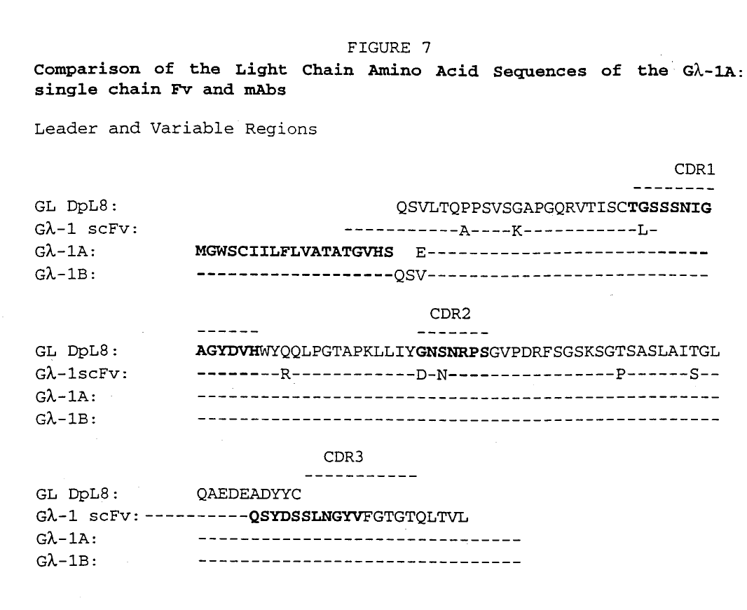

It should be noted that in addition to isolated nucleic acid sequences encoding portions of the altered antibody and antibodies described herein, other such nucleic acid sequences are encompassed by the present invention, such as those complementary to the native CDR-encoding sequences or complementary to the human framework regions surrounding the CDR-encoding regions. Such sequences include all nucleic acid sequences which P50669 by virtue of the redundancy of the genetic code are capable of encoding the same amino acid sequence as given in FIGS. 3 and 4 [SEQ ID NOS: 2 and 4]. FIGS. 6 and 7 [SEQ ID NOS: 5-12] provide representations of such sequences. Other useful DNA sequences encompassed by this invention include those sequences which hybridize under stringent hybridization conditions (See: T. Maniatis et al., Molecular Cloning (A Laboratory Manual), Cold Spring Harbor Laboratory (1982), pages 387 to 389] to the DNA sequences encoding the Gλ-1 antibodies (e.g., sequences of FIGS. 3, 4, 8A-8F through 11 [SEQ ID NOS: 1-4, 13-16]) and which retain the antigen binding properties of those antibodies. An example of one such stringent hybridization condition is hybridization at 4×SSC at 65° C., followed by a washing in 0.1×SSC at 65° C. for an hour. Alternatively an exemplary stringent hybridization condition is in 50% formamide, 4×SSC at 42° C. Preferably, these hybridizing DNA sequences are at least about 18 nucleotides in length, i.e., about the size of a CDR.

V. Altered Immunoglobulin Coding Regions and Altered Antibodies

Altered immunoglobulin coding regions encode altered antibodies which include engineered antibodies such as chimeric antibodies, humanized, reshaped, and immunologically edited human antibodies. A desired altered immunoglobulin coding region contains CDR-encoding regions in the form of scFv regions that encode peptides having the antigen specificity of an RSV antibody, preferably a high affinity antibody such as provided by the present invention, inserted into an acceptor immunoglobulin partner.

When the acceptor is an immunoglobulin partner, as defined above, it includes a sequence encoding a second antibody region of interest, for example, an Fc region. Immunoglobulin partners may also include sequences encoding another immunoglobulin to which the light or heavy chain constant region is fused in frame or by means of a linker sequence. Engineered antibodies directed against functional fragments or analogs of RSV may be designed to elicit enhanced binding with the same antibody.

The immunoglobulin partner may also be associated with effector agents as defined above, including non-protein carrier molecules, to which the immunoglobulin partner may be operatively linked by conventional means.

Fusion or linkage between the immunoglobulin partners, e.g., antibody sequences, and the effector agent may be by any suitable means, e.g., by conventional covalent or ionic bonds, protein fusions, or hetero-bifunctional cross-linkers, e.g., carbodiimide, glutaraldehyde, and the like. Such techniques are known in the art and readily described in conventional chemistry and biochemistry texts.

Additionally, conventional linker sequences which simply provide for a desired amount of space between the second immunoglobulin partner and the effector agent may also be constructed into the altered immunoglobulin coding region. The design of such linkers is well known to those of skill in the art.

In addition, signal sequences for the molecules of the invention may be modified to enhance expression. For example the reshaped human antibody having the signal sequence and CDRs derived from the Fab Gλ-1 heavy chain sequence, may have the original signal peptide replaced with another signal sequence such as the Campath leader sequence [Page, M. J. et al., BioTechnology 9:64-68 (1991)].

An exemplary altered antibody, a reshaped human antibody, contains a variable heavy and the entire light chain peptide or protein sequence having the antigen specificity of Fab Gλ-1, fused to the constant heavy regions C_{H-1}-C_{H-3} derived from a second human antibody.

In still a further embodiment, the engineered antibody of the invention may have attached to it an additional agent. For example, the procedure of recombinant DNA technology may be used to produce an engineered antibody of the invention in which the Fc fragment or C_{H-2}C_{H-3} domain of a complete antibody molecule has been replaced by an enzyme or other detectable molecule (i.e., a polypeptide effector or reporter molecule).

Another desirable protein of this invention may comprise a complete antibody molecule, having full length heavy and light chains, or any discrete fragment thereof, such as the Fab or F(ab′)₂ fragments, a heavy chain dimer, or any minimal recombinant fragments thereof such as an Fᵥ or a single-chain antibody (SCA) or any other molecule with the same specificity as the selected donor Fab Gλ-1. Such protein may be used in the form of an altered antibody, or may be used in its unfused form.

Whenever the immunoglobulin partner is derived from an antibody different from the donor antibody, e.g., any isotype or class of immunoglobulin framework or constant regions, an engineered antibody results. Engineered antibodies can comprise immunoglobulin (Ig) constant regions and variable framework regions from one source, e.g., the acceptor antibody, and one or more (preferably all) CDRs from the donor antibody, e.g., the anti-RSV antibody described herein. In addition, alterations, e.g., deletions, substitutions, or additions, of the acceptor mAb light and/or heavy variable domain framework region at the nucleic acid or amino acid levels, or the donor CDR regions may be made in order to retain donor antibody antigen binding specificity or to reduce potential immunogenicity.

Such engineered antibodies are designed to employ one (or both) of the variable heavy and/or light chains of the RSV mAb (optionally modified as described) or one or more of the below-identified heavy or light chain CDRs. The engineered antibodies of the invention are neutralizing, i.e., they desirably inhibit virus growth in vitro and in vivo in animal models of RSV infection.

Such engineered antibodies may include a reshaped human antibody containing the human heavy and light chain constant regions fused to the RSV antibody functional fragments. A suitable human (or other animal) acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody.

Desirably the heterologous framework and constant regions are selected from human immunoglobulin classes and isotypes, such as IgG (subtypes 1 through 4), IgM, IgA and IgE. The Fc domains are not limited to native sequences, but include mutant variants known in the art that alter function. For example, mutations have been described in the Fc domains of certain IgG antibodies that reduce Fc-mediated complement and Fc receptor binding [see, e.g., A. R. Duncan et al., Nature, 332:563-564 (1988); A. R. Duncan and G. Winter, Nature, 332:738-740 (1988); M.-L. Alegre et al., J. Immunol., 148:3461-3468 (1992); M.-H. Tao et al., J. Exp. Med., 178:661-667 (1993); and V. Xu et al. J. Biol. Chem., 269:3469-2374 (1994)]; alter clearance rate [J.-K. Kim et al., Eur. J. Immunol., 24:542-548 (1994)]; and reduce structural heterogeneity [S. Angal et al., Mol. Immunol. 30:105-108 (1993)]. Also, other modifications are possible such as oligomerization of the antibody by addition of the tailpiece segment of IgM and other mutations [R. I. F. Smith and S. L. Morrison, Biotechnology 12:683-688 (1994); R. I. F. Smith et al., J. Immunol., 154: 2226-2236 (1995)] or addition of the tailpiece segment of IgA [I. Kariv et al., J. Immunol., 157: 29-38 (1996)]. However, the acceptor antibody need not comprise only human immunoglobulin protein sequences. For instance a gene may be constructed in which a DNA sequence encoding part of a human immunoglobulin chain is fused to a DNA sequence encoding a non-immunoglobulin amino acid sequence such as a polypeptide effector or reporter molecule.

The altered antibody thus preferably has the structure of a natural human antibody or a fragment thereof, and possesses the combination of properties required for effective therapeutic use, e.g., treatment of RSV mediated diseases in man, or for diagnostic uses.

It will be understood by those skilled in the art that an altered antibody may be further modified by changes in variable domain amino acids without necessarily affecting the specificity and high affinity of the donor antibody (i.e., an analog). It is anticipated that heavy and light chain amino acids may be substituted by other amino acids either in the variable domain frameworks or CDRs or both. Particularly preferred is the immunological editing of such reconstructed sequences as illustrated in the examples herein.

In addition, the variable or constant region may be altered to enhance or decrease selective properties of the molecules of the instant invention, as described above. For example, dimerization, binding to Fc receptors, or the ability to bind and activate complement [see, e.g., Angal et al., Mol. Immunol, 30:105-108 (1993); Xu et al., J. Biol. Chem, 269:3469-3474 (1994); and Winter et al., EP 307,434-B].

Such antibodies are useful in the prevention and treatment of RSV mediated disorders, as discussed below.

VI. Production of Altered Antibodies And Engineered Antibodies

The resulting reshaped human antibodies of this invention can be expressed in recombinant host cells, e.g., COS, CHO or myeloma cells. A conventional expression vector or recombinant plasmid is produced by placing these coding sequences for the altered antibody in operative association with conventional regulatory control sequences capable of controlling the replication and expression in, and/or secretion from, a host cell. Regulatory sequences include promoter sequences, e.g., CMV promoter, and signal sequences, which can be derived from other known antibodies. Similarly, a second expression vector can be produced having a DNA sequence which encodes a complementary antibody light or heavy chain. Preferably this second expression vector is identical to the first except insofar as the coding sequences and selectable markers are concerned. This ensures as far as possible that each polypeptide chain is functionally expressed. Alternatively, the heavy and light chain coding sequences for the altered antibody may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The production of the antibody which includes the association of both the recombinant heavy chain and light chain is measured in the culture by an appropriate assay, such as an enzyme-linked immunosorbent assay (ELISA) or radioimmunoassay (RIA). Similar conventional techniques may be employed to construct other altered antibodies and molecules of this invention.

Suitable vectors for the cloning and subcloning steps employed in the methods and construction of the compositions of this invention may be selected by one of skill in the art. For example, the conventional pUC series of cloning vectors, may be used. One vector used is pUC19, which is commercially available from supply houses, such as Amersham (Buckinghamshire, United Kingdom) or Pharmacia (Uppsala, Sweden). Any vector, which is capable of replicating readily, has an abundance of cloning sites and selectable genes (e.g., antibiotic resistance), and is easily manipulated, may be used for cloning. Thus, the selection of the cloning vector is not a limiting factor in this invention.

Similarly, the vectors employed for expression of the engineered antibodies according to this invention may be selected by one of skill in the art from any conventional vectors. Preferred vectors include for example plasmids pCD or pCN. The vectors also contain selected regulatory sequences (such as CMV promoters) which direct the replication and expression of heterologous DNA sequences in selected host cells. These vectors contain the above described DNA sequences which code for the engineered antibody or altered immunoglobulin coding region. In addition, the vectors may incorporate the selected immunoglobulin sequences modified by the insertion of desirable restriction sites for ready manipulation.

The expression vectors may also be characterized by genes suitable for amplifying expression of the heterologous DNA sequences, e.g., the mammalian dihydrofolate reductase gene (DHFR). Other preferable vector sequences include a polyadenylation (polyA) signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by,known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the engineered antibodies or altered immunoglobulin molecules thereof. Host cells useful for the cloning and other manipulations of these cloning vectors are also conventional. However, most desirably, cells from various strains of E. coli are used for replication of the cloning vectors and other steps in the construction of altered antibodies of this invention.

Suitable host cells or cell lines for the expression of the engineered antibody or altered antibody of the invention are preferably mammalian cells such as CHO, COS, a fibroblast cell (e.g., 3T3), and myeloid cells, and more preferably a CHO or a myeloid cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., Molecular Cloning (A Laboratory Manual), 2nd edit., Cold Spring Harbor Laboratory (1989).

Bacterial cells may prove useful as host cells suitable for the expression of the recombinant scFvs, Fabs and MAbs of the present invention [see, e.g., Plückthun, A., Immunol. Rev., 130:151-188 (1992)]. The tendency of proteins expressed in bacterial cells to be in an unfolded or improperly folded form or in a non-glycosylated form does not pose as great a concern because Fabs are not normally glycosylated and can be engineered for exported expression, thereby reducing the high concentration that facilitates misfolding. Nevertheless, any recombinant Fab produced in a bacterial cell would be screened for retention of antigen binding ability. If the molecule expressed by the bacterial cell was produced and exported in a properly folded form, that bacterial cell would be a desirable host. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems [see, e.g. Miller et al., Genetic Engineering, 8:277-298, Plenum Press (1986) and references cited therein].

The general methods by which the vectors of the invention may be constructed, the transfection methods required to produce the host cells of the invention, and culture methods necessary to produce the altered antibody of the invention from such host cell are all conventional techniques. Likewise, once produced, the altered antibodies of the invention may be purified from the cell culture contents according to standard procedures of the art, including ammonium sulfate precipitation, affinity columns, column chromatography, gel electrophoresis and the like. Such techniques are within the skill of the art and do not limit this invention.

Yet another method of expression of reshaped antibodies may utilize expression in a transgenic animal. An exemplary systems is described in U.S. Pat. No. 4,873,316. The expression system described in that reference uses the animal's casein promoter and, when transgenically incorporated into a mammal, permits the female to produce the desired recombinant protein in its milk.

Once expressed by the desired method, the engineered antibody is then examined for in vitro activity by use: of an appropriate assay. At present, conventional ELISA assay formats are employed to assess qualitative and quantitative binding of the altered antibody to RSV. Additionally, other in vitro assays and in vivo animal models may also be used to verify neutralizing efficacy prior to subsequent human clinical studies performed to evaluate the persistence of the altered antibody in the body despite the usual clearance mechanisms.

VII. Therapeutic/Prophylactic Uses

This invention also relates to a method of treating humans experiencing RSV-related symptoms which comprises administering an effective dose of antibodies including one or more of the antibodies (altered, reshaped, monoclonal, etc.) described herein or fragments thereof.

The therapeutic response induced by the use of the molecules of this invention is produced by binding to RSV and thus subsequently blocking RSV propagation. Thus, the molecules of the present invention, when in preparations and formulations appropriate for therapeutic use, are highly desirable for those persons experiencing RSV infection. For example, longer treatments may be desirable when treating seasonal episodes or the like. The dose and duration of treatment relates to the relative duration of the molecules of the present invention in the human circulation, and can be adjusted by one of skill in the art depending upon the condition being treated and the general health of the patient.

The altered antibodies, antibodies and fragments thereof of this invention may also be used alone or in conjunction with other antibodies, particularly human or humanized mAbs reactive with other epitopes on the F protein or other RSV target antigens as prophylactic agents.

The mode of administration of the therapeutic and prophylactic agents of the invention may be any suitable route which delivers the agent to the host. The altered antibodies, antibodies, engineered antibodies, and fragments thereof, and pharmaceutical compositions of the invention are particularly useful for parenteral administration, i.e., subcutaneously, intramuscularly, intravenously, or intranasally.

Therapeutic and prophylactic agents of the invention may be prepared as pharmaceutical compositions containing an effective amount of the altered antibody of the invention as an active ingredient in a pharmaceutically acceptable carrier. An aqueous suspension or solution containing the antibody, preferably buffered at physiological pH, in a form ready for injection is preferred. The compositions for parenteral administration will commonly comprise a solution of the engineered antibody of the invention or a cocktail thereof dissolved in an pharmaceutically acceptable carrier, preferably an aqueous carrier. A variety of aqueous carriers may be employed, e.g., 0.4% saline, 0.3% glycine, and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 80 mg, or more preferably, about 5 mg to about 75 mg, of an engineered antibody of the invention. Similarly, a pharmaceutical composition of the invention for intravenous infusion could be made up to contain about 250 ml of sterile Ringer's solution, and about 1 to about 75 and preferably 5 to about 50 mg/ml of an engineered antibody of the invention. Actual methods for preparing parenterally administrable compositions are well known or will be apparent to those skilled in the art and are described in more detail in, for example, Remington's Pharmaceutical Science, 15th ed., Mack Publishing Company, Easton, Pa.

It is preferred that the therapeutic and prophylactic agents of the invention, when in a pharmaceutical preparation, be present in unit dose forms. The appropriate therapeutically effective dose can be determined readily by those of skill in the art. To effectively treat an inflammatory disorder in a human or other animal, one dose of approximately 0.1 mg to approximately 20 mg per 70 kg body weight of a protein or an antibody of this invention should be administered parenterally, preferably i.v. or i.m. (intramuscularly). Such dose may, if necessary, be repeated at appropriate time intervals selected as appropriate by a physician.

The altered antibodies and engineered antibodies of this invention may also be used in diagnostic regimens, such as for the determination of RSV mediated disorders or tracking progress of treatment of such disorders. As diagnostic reagents, these altered antibodies may be conventionally labeled for use in ELISAs and other conventional assay formats for the measurement of RSV levels in serum, plasma or other appropriate tissue, or the release by human cells in culture. The nature of the assay in which the altered antibodies are used are conventional and do not limit this disclosure.

The antibodies, altered antibodies or fragments thereof described herein can be lyophilized for storage and reconstituted in a suitable carrier prior to use. This technique has been shown to be effective with conventional immunoglobulins and art-known lyophilization and reconstitution techniques can be employed.

The following examples illustrate various aspects of this invention including the construction of exemplary engineered antibodies and expression thereof in suitable vectors and host cells, and are not to be construed as limiting the scope of this invention. All amino acids are identified by conventional three letter or single letter codes. All necessary restriction enzymes, plasmids, and other reagents and materials were obtained from commercial sources unless otherwise indicated. All general cloning ligation and other recombinant DNA methodology were as performed in T. Maniatis et al., cited above, or Sambrook et al., cited above.

### EXAMPLE 1

### Isolation of Gλ-1 scFv-1

Single chain (sc) Fv libraries were prepared from an individual purposely exposed to RSV and selected against recombinant RSV F-protein following described procedures [R. H. Jackson et al, in Protein Engineering, A Practical Approach, A. R. Rees et al eds, Oxford University Press, chapter 12, pp. 277-301, 1992; H. R. Hoogenboom et al., Nucl. Acid Res., 19: 4133-4137 (1991); J. D. Marks et al., J. Mol. Biol., 222: 581-597 (1991)]. Briefly, lymphocytes were isolated from a blood sample taken 15 days post exposure. RNA isolated from the lymphocytes was used for preparation of scFv encoding repertoires for phage display. Sets of V-region primers were paired with constant region primers for heavy chain domain 1 IgG and IgM and light chain C-κ and C-λ and then linked in a scFv VH-VL orientation with a 15 amino acid spacer (glycine₄-serine)₃ [SEQ ID NO: 21] by overlap PCR [see J. D. Marks et al., cited above, for description of the primers].

The resulting four scFv repertoires (V-κ with IgG and IgM, V-λ with IgG and IgM) were cloned into a phagemid vector similar to pHEN1 [H. R. Hoogenboom et al., cited above] resulting in fusion of the scFvs to gene III of phage fd. The vector was then transformed into E. coli (e.g., strain TG1) by electroporation to yield the corresponding phagemid libraries.

Phage libraries displaying the scFv-gene 3 fusions were prepared by infection of each of the plasmid libraries with the M13K07 helper phage [R. H. Jackson, cited above] and were individually subjected to 2 rounds of panning against recombinant F-protein coated onto plastic. In the first round, 10¹¹ phage in 2.5 ml phosphate buffered saline (PBS)/2% Marval™ non-fat dry milk were incubated for 90 minutes in a tube coated with 5 μg/ml of F-protein [described in P. Tsui et al, J. Immunol., 157:772-780 (1996)] followed by 1 wash with 10×PBS/0.05% Tween 20 and a second wash with 10×PBS alone. Bound phage were eluted with 10 mM triethylamine and the eluate was neutralized with 1 M Tris-HCl, pH 7.4. The eluted phage were amplified and subjected to a similar second round of panning, except that the concentration of F-protein for coating was 2 μg/ml and the wash buffer contained 20×PBS.

E. coli were infected with the eluted phage and 96 colonies from each starting library were superinfected with helper phage and screened for F-protein binding activity. Only four positive clones were obtained from the 2 IgM libraries, whereas 41 positives were observed for the IgG libraries. By partial sequence analysis, all of the clones carried one of three different heavy chains. Complete sequences were obtained for the heavy and light chain V-regions for six clones, all from the IgG libraries.

Serial dilutions of titered phage stocks of each of these six clones were tested by ELISA for binding to recombinant F-protein and to RSV infected cell lysate. All showed binding to F-protein with the phage designated Gλ-1 showing the best activity. However, Gλ-1 and three other clones showed little binding to the RSV lysate.

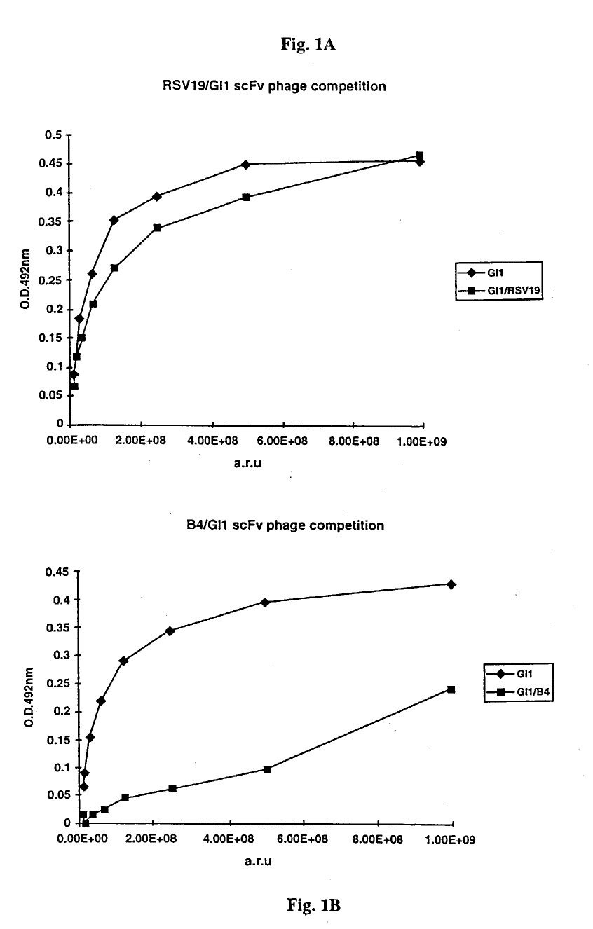

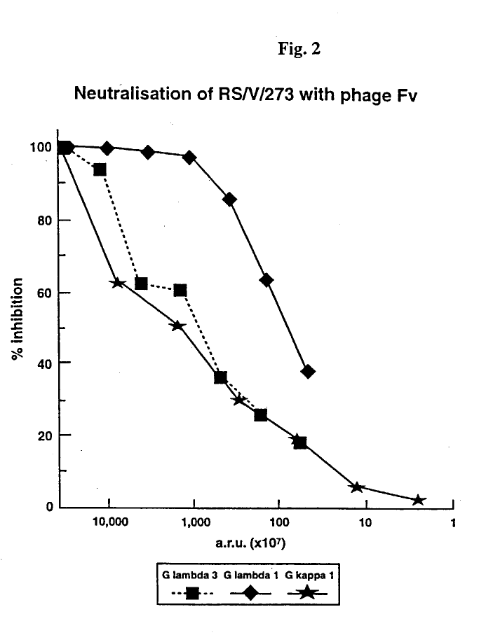

Three clones: Gλ-1, Gλ-3 (lysate binding positive), and Gκ-1 (lysate binding negative), where “κ” and “λ” designate the class of the light chain, were characterized further for competition of their binding by F-protein specific neutralizing monoclonal antibodies, and their ability to inhibit virus infection. The neutralizing mAbs RSV19 and B4 described in International patent publication No. WO92/04381, published Mar. 19, 1992, and International patent publication No. WO93/20210, published Oct. 14, 1993, recognize distinct epitopes on the F-protein. Gκ-1 was strongly inhibited by both antibodies. Gλ-1 was significantly inhibited by B4 only. Gκ-3 was not inhibited by either antibody (shown for Gλ-1 only; see FIGS. 1A and 1B). In initial assays (Table I, experiments 1-3), all three clones showed neutralizing activity in vitro, with Gλ-1 being the most potent (FIG. 2, a graph of experiment 2), while control wild-type phage (M13K07) not displaying scFv had no effect.

To address the possibility that neutralization might result just from phage coating of virus, irrespective of epitope, a phage preparation of the non-neutralizing Fab 5-16 was tested in the same assay. In three out of four assays, this preparation also showed good neutralization activity, as did the control phage in two of these assays (Table I, experiments 4-7). This confounding observation of variable neutralization by both Fab 5-16 and control M13K07 phage rendered the viral neutralization studies inconclusive.

TABLE I

| | Virus Neutralization (IC₅₀ × 10⁻⁷)¹ | | | | | | |
|---|---|---|---|---|---|---|---|
| | (aru or kru/ml)² | | | | | | |
| Phage | Experiment # | | | | | | |
| Sample | 1 | 2 | 3 | 4 | 5 | 6 | 7 |
| Gκ-1 a | 1,600 | | <300 | | | | |
| b | | | | <10 | <7 | | |
| Gλ-1 a | | 80 | <300 | | | | |
| b | | | | 8.1 | 11 | | |
| c | | | | | | | 120 |
| Gλ-3 a | | 900 | <300 | 180 | | | |
| b | | | | | <7 | 10 | |
| c | | | | | | | 730 |
| M13K07a | | | >10⁵ | >10⁵ | | >5,000 | |
| b | | | | | +all dil. | +all dil. | >10⁴ |
| Fab 5-19a | | | | >10⁵ | 40 | 180 | |
| b | | | | | | | 3.5 |

Legend:
¹Assay according to M. J. Cannon, J. Virol. Meth., 16: 293-301. Virus at 100 infectious centers/well was incubated with dilutions of the indicated phage for 1 hr and then added to susceptible cells for 3 hr. The virus/phage solution was aspirated and replaced with fresh medium and the cells were incubated overnight before peroxidase staining for virus infected cells.
²aru = ampicillin resistance units, a measure of phagmid containing particles.
kru = kanamycin resistance units, a measure of particles containing the phage genome (for the M13K07 control only).

In the face of these results, made more ambiguous by the dependence of all assays on phage stocks verses antibody proteins of known concentration, Gλ-1 was selected as the most likely candidate for a potent neutralizing antibody based on (1) its apparent better binding to F-protein, (2) its selective inhibition of binding by the B4 antibody, and (3) its suggested activity over background in the virus neutralization assay.

### EXAMPLE 2

### Conversion of Gλ-1 scFV To mAb Version A

The DNA and encoded protein sequences of the VH and VL regions of Gλ-1 are shown in FIGS. 3 [SEQ ID NOS: 1 and 2] and 4 [SEQ ID NOS: 3 and 4], respectively. For expression in mammalian cells, the heavy chain variable region and the light chain variable region from the Gλ-1 plasmid were cloned into derivatives of plasmid pCDN [Nambi, A. et al., Mol. Cell. Biochem., 131:75-86 (1994)] in which the expression of the antibody chain is driven by the cytomegalovirus promoter (CMV) promoter. Plasmid pCD-HC68B is used for expressing full length heavy chains and plasmid pCN-HuLC, for expressing full length light chains.

In the initial constructs, changes in the sequence at the amino terminus were introduced by the PCR primers used for cloning the light chain and heavy chain variable regions from plasmid Gλ-1. In these constructs, the peptide signal sequence for both the heavy and light chains is derived from the Campath light chain [M. J. Page et al., Biotechnology 9: 64-68 (1991)]. The heavy chain of Gλ-1 was PCR amplified from Gλ-1 phagemid DNA, using primers for the amino terminus and framework 4 of the variable region. The resulting PCR fragment was cut with XhoI (site introduced by the amino terminus primer) and BstEII (naturally occurring site in framework 4), and cloned into an intermediate vector, F4HCV, at the XhoI/BstEII sites.

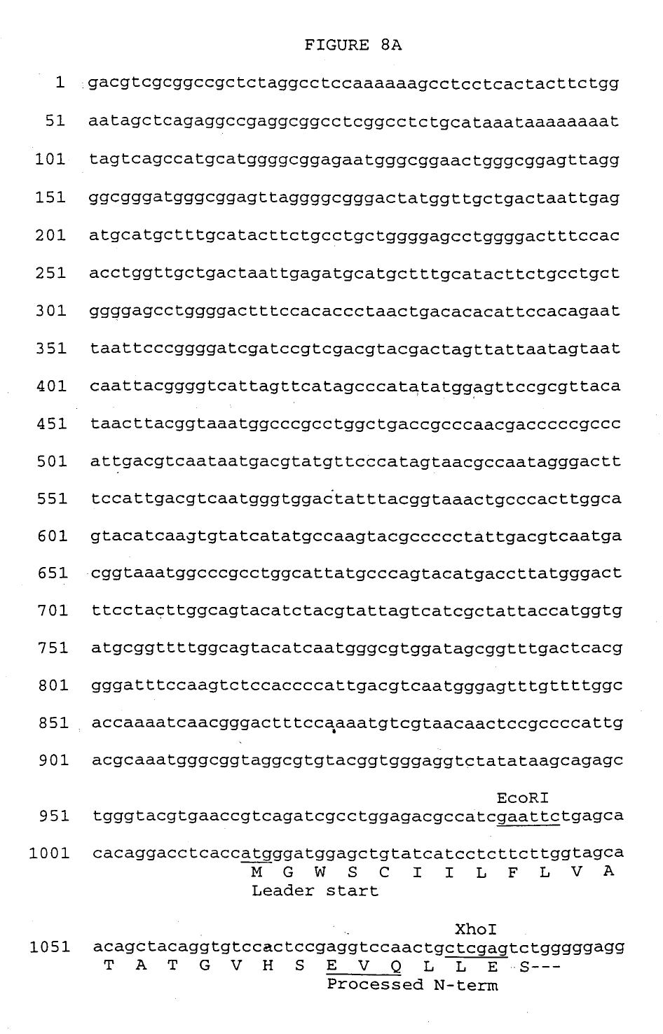

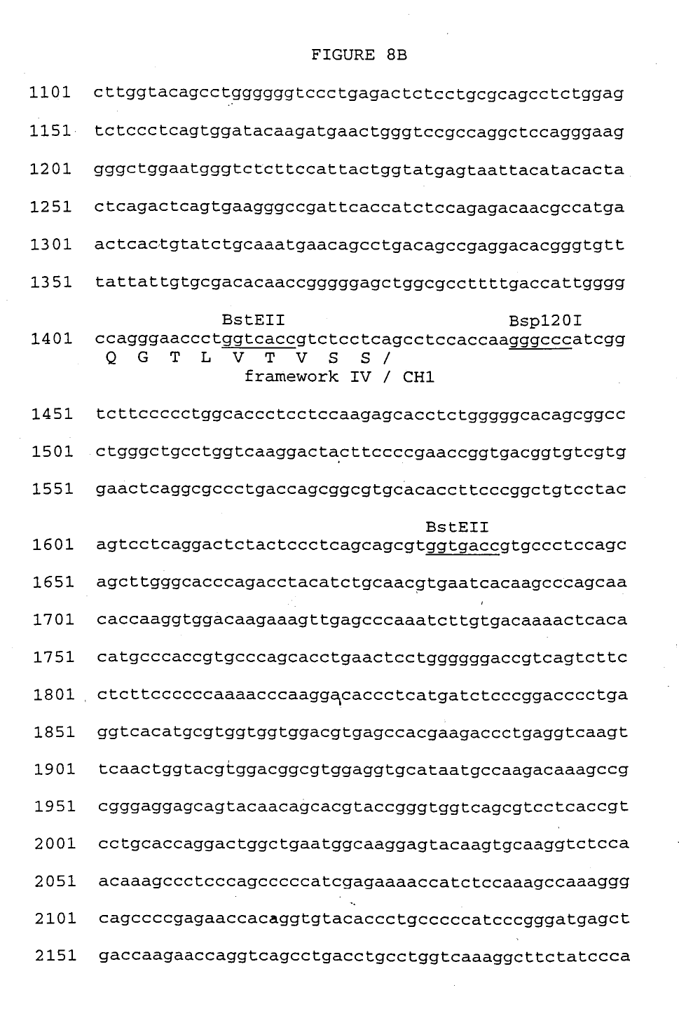

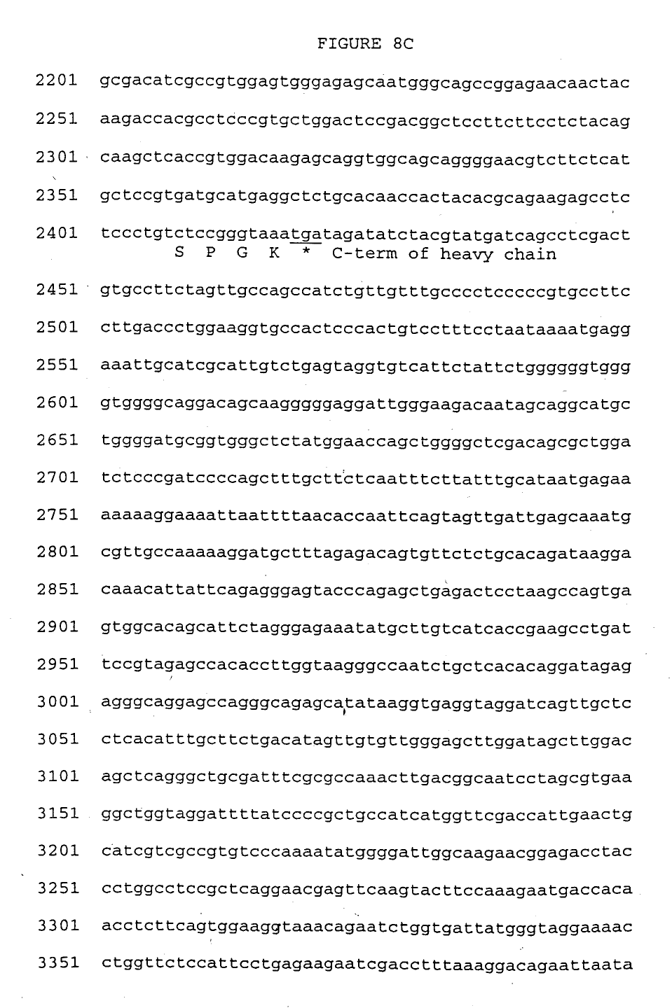

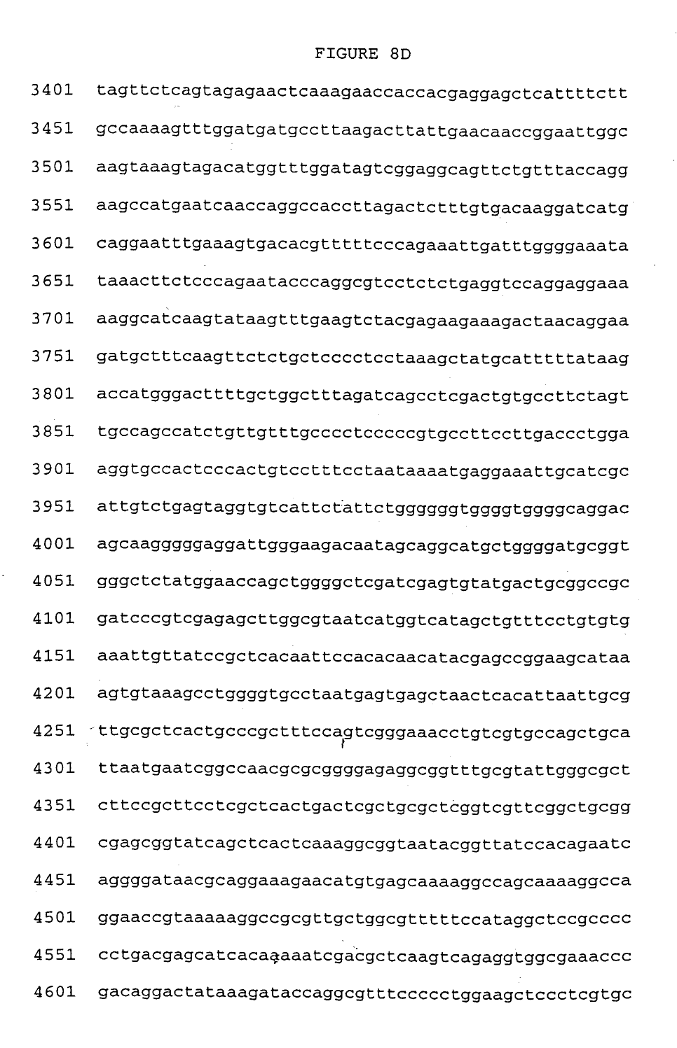

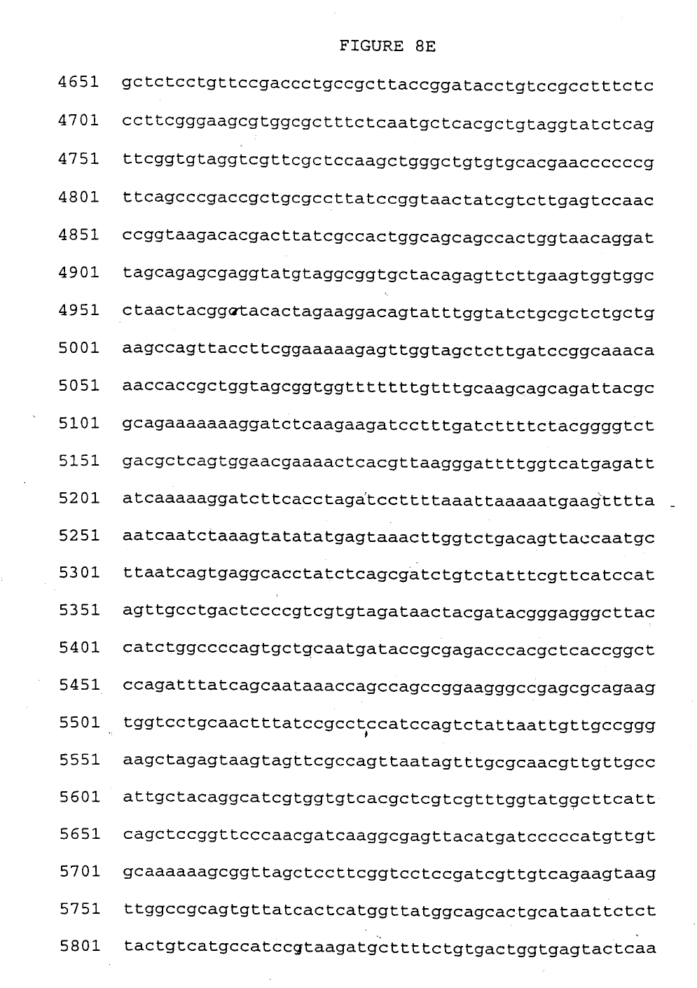

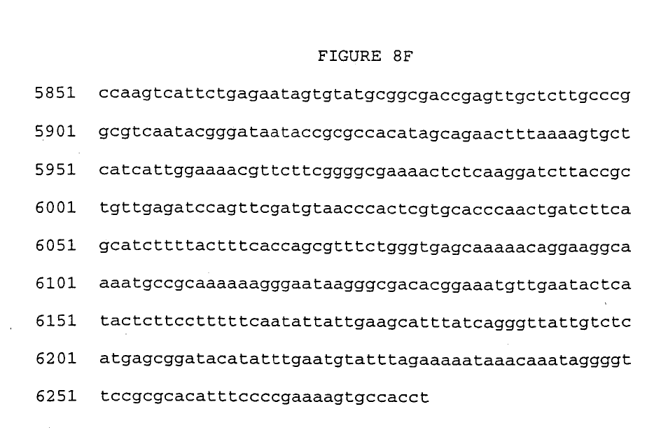

This cloning grafted the variable region of Gλ-1 onto the constant region of another anti-RSV heavy chain 194-F4 [cloned at SmithKline Beecham from a human hybridoma]. This intermediate clone was cut with XhoI and Bsp120I, and introduced into the same sites in pCD-HC68B. The XhoI site is introduced at the amino terminus by the PCR primer and, when cloned into pCD-HC68B at the same site is preceded in frame by the Campath leader sequence. The Bsp120I site is a naturally occurring, highly conserved sequence at the beginning of the C_{H-1} domain, and when cloned into pCD-HC68B at the same site, is in frame with the remaining sequence for the C_{H-1} through C_{H-3} regions of human IgG₁. In the resulting construct, Gλ-1Apcd (FIGS. 8A-8F [SEQ ID NO: 13]), the amino acids immediately following the Campath leader are EVQLLE [SEQ ID NO: 17], where the residues LE are encoded by the nucleotide sequence for the XhoI cloning site.

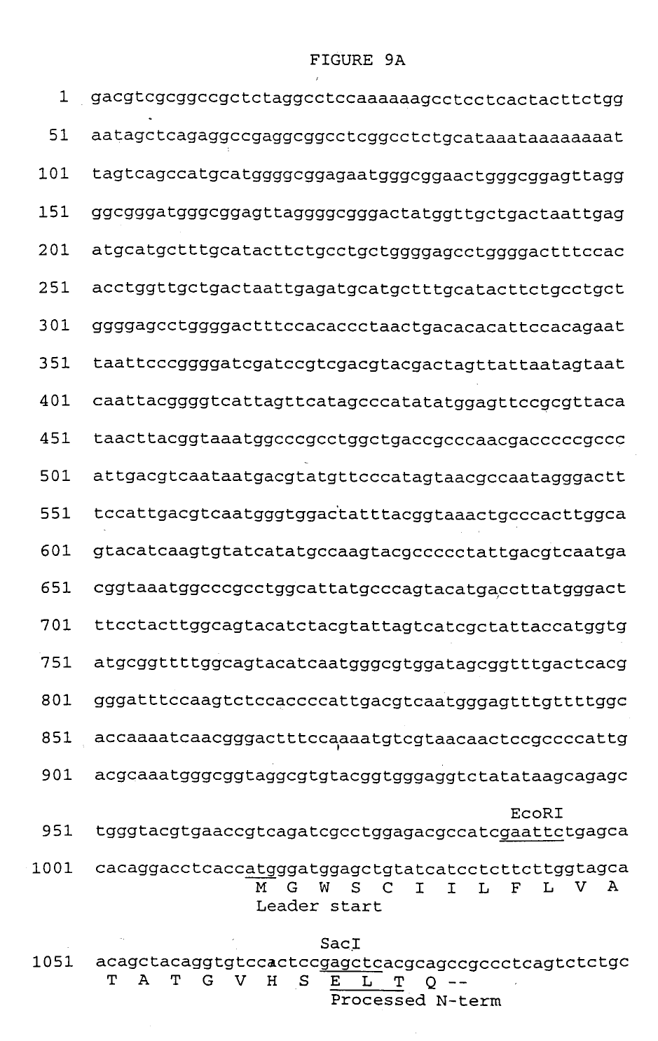

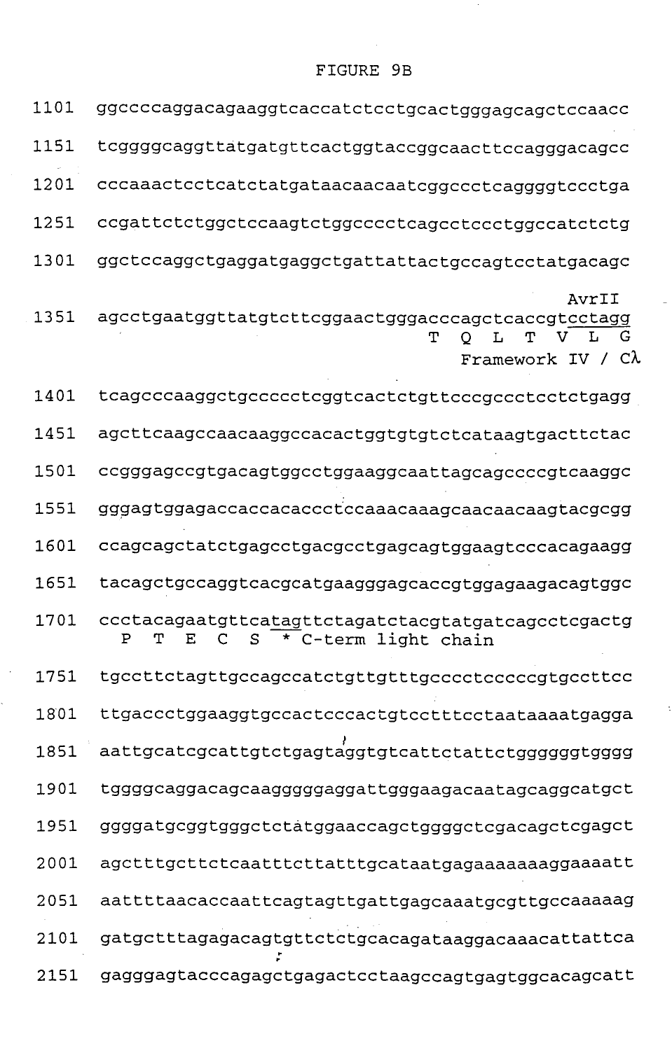

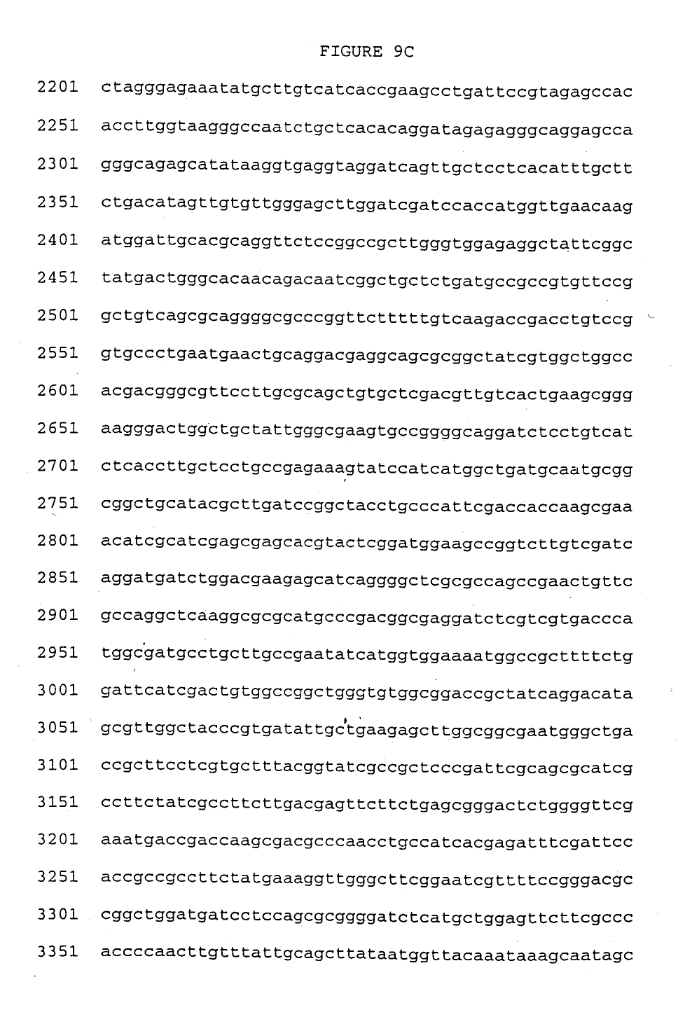

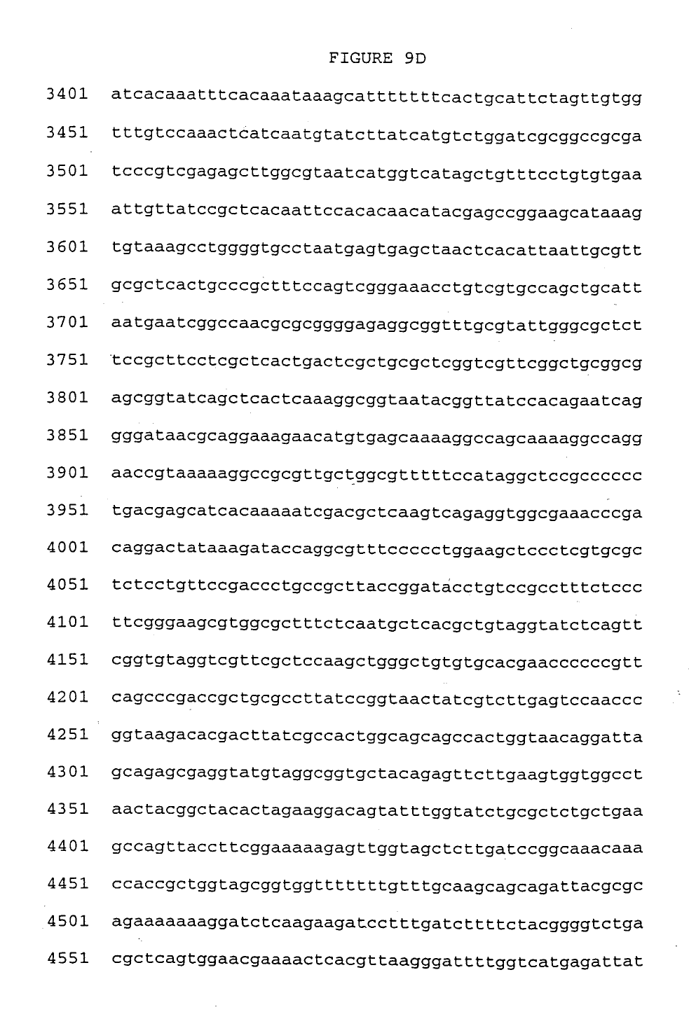

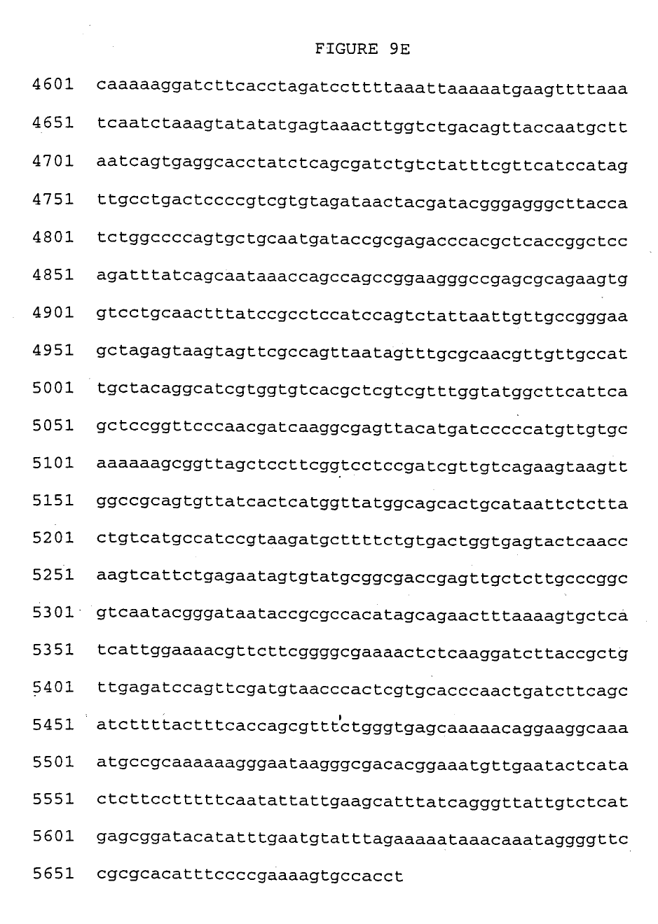

The light chain of Gλ-1 was PCR amplified from the Gλ-1 phagemid DNA, using primers for the amino terminus and framework 4 of the variable region. The resulting PCR fragment was cut with SacI (site introduced by the amino terminus primer) and AvrII (naturally occurring site in framework 4), and cloned into 43-1pcn at the SacI/AvrII sites. This cloning grafted the variable region of Gλ-1, in frame, onto the constant region of another anti-RSV lambda light chain 43 [P. Tsui et al., J. Immunol., 157: 772-780 (1996)], which had been cloned at SmithKline Beecham from a combinatorial library derived from RNA isolated from human spleen. The SacI site is introduced at the amino terminus by the PCR primer and, when cloned into 43pcn at the same site, is preceded in frame by the Campath leader sequence. The first two amino acids of the mature light chain are therefore deleted. In the resulting construct, Gλ-1Apcn (FIGS. 9A-9E [SEQ ID NO: 14]), the first two amino acids immediately following the leader are EL, where the residues EL are encoded by the nucleotide sequence for the SacI cloning site.

The nucleotide sequences of the plasmids Gλ-1Apcd and Gλ-1Apcn are shown in FIGS. 8A-8F [SEQ ID NO: 13] and 9A-9E [SEQ ID NO: 14] respectively. This set of vectors was used to produce antibody Gλ-1A in COS cells and in CHO cells.

### EXAMPLE 3

### Cloning of the Corrected Gλ-1 Heavy and Light Chains

In cloning the variable region of the Gλ-1 heavy chain from the single chain Fv (scFv) format into the full length format, the fifth amino acid at the amino terminus was changed from Val to Leu, for cloning purposes. To correct this change, PCR primers were designed for the amino terminus of the Gλ-1 heavy chain cloned into pCD, which reverted the fifth amino acid back to Val. The correction was introduced via the PCR overlap technique using the correction primers and primers annealing to sequences within the CMV promoter and the C_{H-2} constant region as the outside 5′ and 3′ primers, respectfully. The final PCR product was digested with restriction enzymes, EcoRI and Bsp120I, and cloned into the Gλ-1Apcd vector at the same sites to create Gλ-1Bpcd.

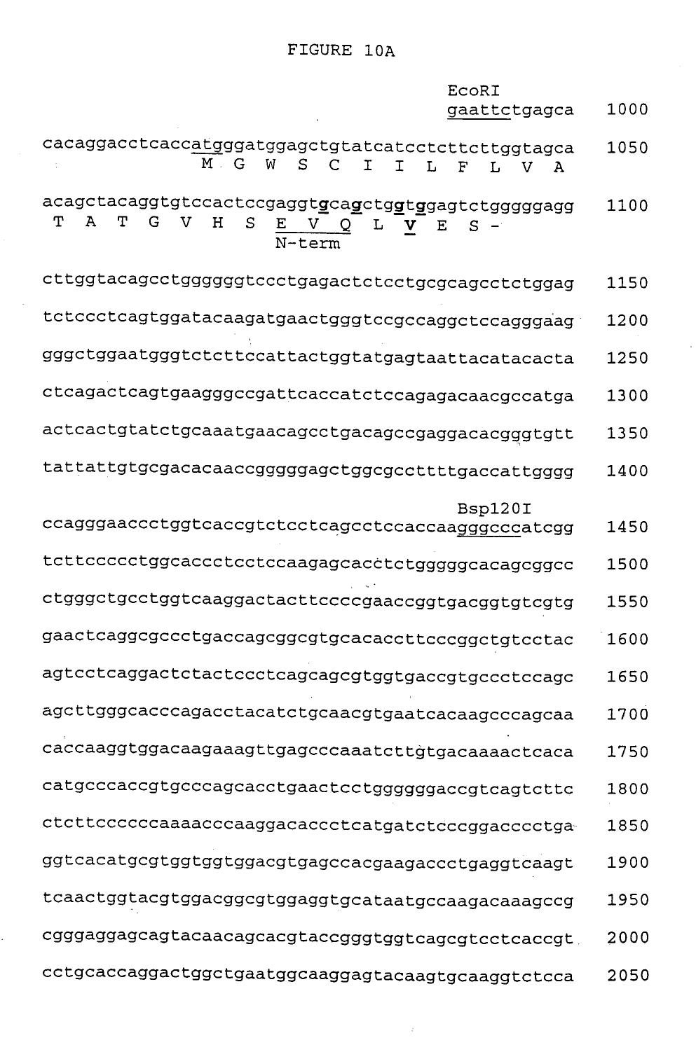

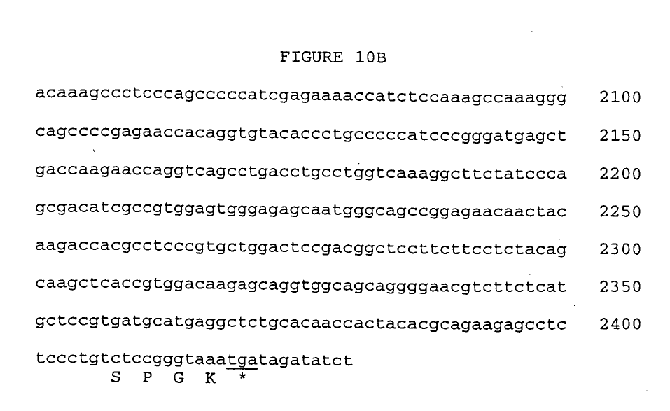

The final construct was sequenced to verify that the amino terminus of the heavy chain had been corrected from EVQLLE [SEQ ID NO: 17] to EVQLVE [SEQ ID NO: 18] (see FIG. 6). The nucleotide sequence of coding region for the corrected heavy chain, Gλ-1B, is shown in FIGS. 10A-10B [SEQ ID: NO: 15].

In cloning the variable region of the Gλ-1 light chain from the scFv format into the full length format, changes were introduced at the amino terminus for cloning purposes. Specifically, the first 2 amino acids (Gln and Ser) of the light chain were deleted and the third amino acid was changed from Val to Glu. To correct these changes, PCR primers were designed for the amino terminus of the Gλ-1 light chain cloned into pCN, which replaced the two deleted amino acids (Gln and Ser) and reverted the third amino acid back to Val. The corrections were introduced via the PCR overlap technique using the correction primers and primers annealing to sequences within the CMV promoter and the λ constant region as the outside 5′ and 3′ primers, respectfully. The final PCR product was digested with restriction enzymes, EcoRI and AvrII and cloned into the Gλ-1Apcn vector at the same sites to create Gλ-1Bpcn.

The final construct was sequenced to verify that the amino terminus of the light chain had been corrected from --EL to QSVL (amino acids 1-4 of SEQ ID NO: 10).

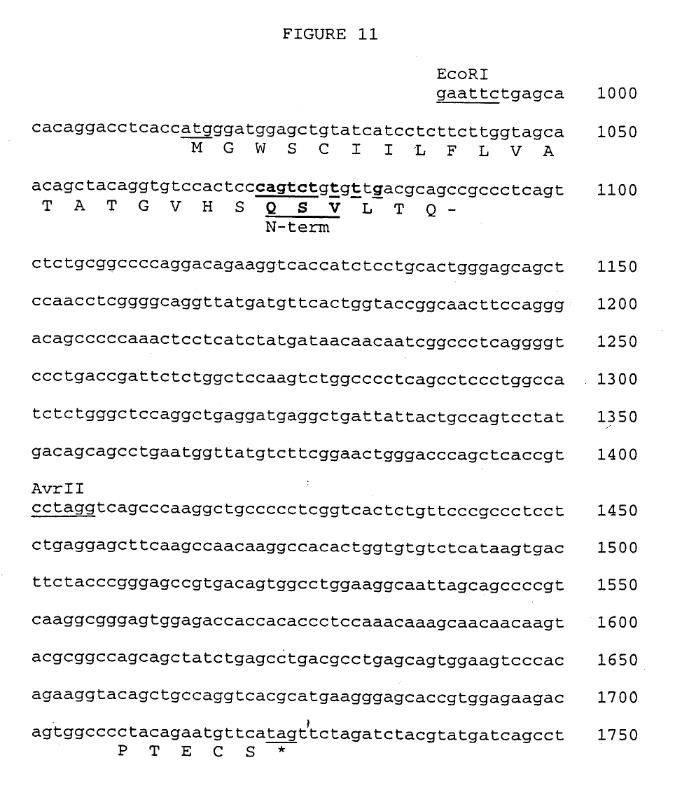

The nucleotide sequence of coding region for the corrected light chain, Gλ-1B, is shown in FIG. 11 [SEQ ID NO: 16]. This vector Gλ-1Bpcn, was used with Gλ-1Bpcd to produce antibody Gλ-1B, in COS cells and in CHO cells.

### EXAMPLE 4

### Production of Gλ-1 mABs in Mammalian Cells

For initial characterization, the mAb constructs for each version, Gλ-1A heavy and light chain, Gλ-1B heavy and light chain, were expressed in COS cells essentially as described in Current Protocols in Molecular Biology, eds F. M. Ausubel et al., 1988, John Wiley & Sons, vol. 1, section 9.1. On day 1 after the transfection, the culture growth medium was replaced with a serum-free medium [SmithKline Beecham] which was changed on day 3. Similar satisfactory results are obtained using a publicly available medium, DMEM supplemented with ITS™ Premix, an insulin, transferrin, selenium mixture (Collaborative Research, Bedford, Mass.) and 1 mg/ml bovine serum albumin (BSA).

The mAb was prepared from the day 3+day 5 conditioned medium by standard protein A affinity chromatography methods (e.g., as described in Protocols in Molecular Biology) using, for example, Prosep A affinity resin (Bioprocessing Ltd., UK).

To produce larger quantities of the Gλ-1B mAB (100-200 mgs), the vectors were introduced into a proprietary CHO cell system. However, similar results will be obtained using dhfr⁻ CHO cells as previously described [P. Hensley et al., J. Biol. Chem., 269:23949-23958 (1994)]. Briefly, a total of 30 μg of linearized plasmid DNA. (15 μg each of the A or B set of heavy chain and light chain vectors) is electroporated into 1×10⁷ cells. The cells are initially selected in nucleoside-free medium in 96 well plates. After three to four weeks, media from growth positive wells is screened for human immunoglobulin using an ELISA assay. The highest expressing colonies are expanded and selected in increasing concentrations of methotrexate for amplification of the transfected vectors. The antibody is purified from conditioned medium by standard procedures using protein A affinity chromatography (Protein A sepharose, Pharmacia) followed by size exclusion chromatography (Superdex 200, Pharmacia).

The concentration and the antigen binding activity of the eluted antibody are measured by ELISA. The antibody containing fractions are pooled and further purified by size exclusion chromatography. As expected for any such antibody, by SDS-PAGE, the predominant protein product migrated at approximately 150 kd under non-reducing conditions and as two bands of 50 and 25 kd under reducing conditions. For antibody produced in CHO cells, the purity was >90%, as judged by SDS-PAGE, and the concentration was accurately determined by amino acid analysis.

### EXAMPLE 5

### Binding of the Gλ-1 mABs To Recombinant F Protein

Binding of the Gλ-1 mABs to recombinant F protein was measured in a standard solid phase ELISA. Antigen diluted in PBS pH 7.0 was adsorbed onto polystyrene round-bottom microplates (Dynatech, Immunolon II) for 18 hours. Wells were then aspirated and blocked with 0.5% boiled casein (BC) in PBS containing 1% Tween 20 (PBS/0.05% BC) for two hours. Antibodies (50 μl/well) were diluted to varying concentrations in PBS/0.5% BC containing 0.025% Tween 20 and incubated in antigen coated wells for one hour. Plates were washed three times with PBS containing 0.05% Tween 20, using a Titertek 320 microplate washer, followed by addition of HRP-labelled protein A/G (50 μl) diluted 1:5000. After washing three times, TMBlue substrate (TSI, #TM102) was added and plates were incubated an additional 15 minutes. The reaction was stopped by addition of 1 N H₂SO₄ and absorbance read at 450 nm using a Biotek ELISA reader.

The antigen binding epitope of the Gλ-1 mABs was examined in a competition ELISA. The Gλ-1 mABs were mixed with increasing concentrations of RSMU19 or B4, two potent neutralizing mAbs [Tempest et al., Biotech., 9: 266-271 (1991); Kennedy et al., J. Gen. Virol., 69: 3023-3032 (1988)] and added to F protein-coated wells. The epitope regions recognized by mAbs RSMU19 and B4 are quite distinct from each other as previously described in Arbiza et al., J. Gen. Virol., 73:2225-2234 (1992). The concentration of the Gλ-1 mABs used in competition studies was determined previously to give 90% maximal binding to F antigen. Binding of the Gλ-1 mABs in the presence of other mABs was detected using HRP-labelled goat anti-human IgG. The reaction was developed as stated above.

The Gλ-1 mABs demonstrated potent binding to recombinant F (rF) protein by ELISA (ECso for mAB B=2.6 ng/ml). Binding of the Gλ-1 mABs to rF protein was inhibited by mAb B4, for which the F protein amino acids critical for antigen recognition are amino acids 268, 272 and 275 of SEQ ID NO: 20). Binding of the Gλ-1 mABs to rF protein was not inhibited by mAb RSMU19, for which F protein amino acid 429 of SEQ ID NO: 20 is critical for antigen recognition. These results indicate that residues in the region of amino acids 255-275 of the F protein [SEQ ID NO: 20] are critical for Gλ-1 mAB recognition.

### EXAMPLE 6

### In vitro Fusion-Inhibition Activity of the Gλ-1 MABs

The ability of the Gλ-1 mABs to inhibit virus-induced cell fusion was determined using a modification of the in vitro microneutralization assay [Beeler et al., J. Virol., 63:2941-2950 (1989)]. In this assay, 50 μl of RS Long strain virus (10-100 TCID₅₀/well [American Type Culture Collection ATCC VR-26] were mixed with 0.1 ml VERO cells (5×10³/well) [ATCC CCL-81] in Minimum Essential Media (MEM) containing 2% fetal calf serum (FCS), for 4 hours at 37° C., 5% CO₂. Serial two-fold dilutions (in quadruplicate) of mAB (50 μl) were then added to wells containing virus-infected cells. Control cultures contained cells incubated with virus only (positive virus control) or cells incubated with media alone.

Cultures were incubated at 37° C. in 5% CO₂ for 6 days at which time cytopathic effects (CPE) in virus control wells were >90%. Microscopic examination for cytopathic effects were confirmed by ELISA. Media was aspirated from cultures and replaced with 50 μl of 90% methanol containing 0.6% H₂O₂. After 10 minutes, fixative was aspirated and plates were air dried overnight. Viral antigen was detected in the fixed cultures using 1 μg/ml biotinylated RSCHB4 (a human Fc derivative of the bovine B4 mAb [SmithKline Beecham]), followed by HRP-labelled streptavidin (Boehringer-Mannheim) diluted 1:10,000. The reaction was developed using TMBlue and stopped by addition of 1N H₂SO₄. Absorbance was measured at 450 nm (O.D.₄₅₀).

Fusion-inhibition titers were defined as the concentration of antibody which caused a 50% reduction in ELISA signal (ED₅₀) as compared to virus controls. Based on the curve generated in the ELISA by the standard virus titration, a 50% reduction in O.D.₄₅₀ corresponded to ≧90% reduction in virus titer. Calculation of the 50% point was based on regression analysis of the dose titration.

The Gλ-1 mABs demonstrated potent in vitro fusion-inhibition activity against type A RS Long strain virus (ED₅₀ for mAB B of 0.51±0.38 μg/ml). In this in vitro fusion-inhibition assay, Gλ-1 mAB B was more active than the humanized mAB RSHZ19 (ED₅₀ of 0.4-3.0 μg/ml) [Wyde et al., Pediatr. Res., 38(4):543-550] in comparative assays.

### EXAMPLE 7

### In Vivo Activity of Gλ-1 mAB B: Prophylaxis And Therapy In Balb/c Mouse Model

Balb/c mice (5/group) were inoculated intraperitoneally with doses ranging from 0.06 mg/kg to 5 mg/kg of Gλ-1 mAB B either 24 hours prior (prophylaxis) or 4 days after (therapy) intranasal infection with 10⁵ PFU of the A2 strain of human RSV. Mice were sacrificed 5 days after infection. Lungs were harvested and homogenized to determine virus titers.

Virus was undetectable in the lungs of mice treated prophylactically with ≧1.25 mg/kg Gλ-1 mAB B either prophylactically or therapeutically. See Table II below. Significant viral clearance (2-3 log₁₀) was also achieved in animals receiving 0.31 mg/kg Gλ-1 mAB B either prophylactically or therapeutically.

TABLE II
Gλ-1 mAB B Prophylaxis and Therapy in Balb/c Mice

| | Dose | Lung Virus Titer | (log₁₀/g lung) |
|---|---|---|---|
| Treatment | (mg/kg) | Prophylaxis | Therapy |
| Gλ-1 mAB B | 5 | <1.7 | <1.7 |
| | 1.25 | <1.7 | <1.7 |
| | 0.31 | 1.8 ± 0.3 | 2.9 ± 0.4 |
| | 0.06 | 4.3 ± 0.7 | 4.5 ± 0.3 |
| PBS | — | 4.8 ± 0.7 | 4.7 ± 0.2 |

The Gλ-1 mABs have potent antiviral activity in vitro against a broad range of native RSV isolates of both type A and B, and show prophylactic and therapeutic efficacy in vivo in animal models. Thus, the Gλ-1 MABs are candidates for therapeutic, prophylactic, and diagnostic application in man.

Numerous modifications and variations of the present invention may be made by one of skill in the art in view of the invention described herein. Such modifications are believed to be encompassed by the specification and claims of the present invention. All references cited above are incorporated by reference herein.

1. A human monoclonal antibody and functional fragments thereof, specifically reactive with an F protein epitope of Respiratory Syncytial Virus and capable of neutralizing infection by said virus selected from the group consisting of Gλ-1A and Gλ-1B.

2. The monoclonal antibody according to claim 1 which comprises the light chain amino acid sequence of FIG. 3 SEQ ID NO: 2 and the heavy chain amino acid sequence of FIG. 4 SEQ ID NO: 4.

3. The monoclonal antibody according to claim 1 which comprises the light chain amino acid sequence encoded by the DNA sequence of FIG. 11 SEQ ID NO: 16 and the heavy chain amino acid sequence encoded by the DNA sequence of FIGS. 10A-10B SEQ ID NO: 15.

4. The monoclonal antibody according to claim 1 wherein said fragment is selected from the group consisting of Fv, Fab and F(ab′)₂.

5. An isolated nucleic acid molecule selected from the group consisting of:
(a) a nucleic acid sequence encoding any of the human monoclonal antibodies, altered antibodies and CDRs of any of the claims 1-4;
(b) a nucleic acid complementary to any of the sequences in (a); and
(c) a nucleic acid sequence of 18 or more nucleotides capable of hybridizing to the CDRs of any of claims 1-4 under stringent conditions.

6. The isolated nucleic acid molecule according to claim 5 comprising the sequences of FIGS. 8A-8F and 9A-9E SEQ ID NOS: 13 and 14, or FIGS. 10A-10B and 11 SEQ ID NOS: 15 and 16.

7. A recombinant plasmid comprising the nucleic acid sequences of any of claims 5 or 6.

8. A host cell comprising the plasmid of claim 7.

9. A process for the production of a human antibody specific for RSV comprising culturing the host cell of claim 8 in a medium under suitable conditions of time temperature and pH and recovering the antibody so produced.

10. A method of detecting RSV comprising contacting a source suspected of containing RSV with a diagnostically effective amount of the monoclonal antibody of claim 1 and determining whether the monoclonal antibody binds to the source.

11. A method for providing passive immunotherapy to RSV disease in a human, comprising administering to the human an immunotherapeutically effective amount of the monoclonal antibody of claim 1.

12. The method according to claim 11 wherein the passive immunotherapy is provided prophylactically.

13. A pharmaceutical composition comprising at least one dose of an immunotherapeutically effective amount of the monoclonal antibody of claim 1 in a pharmaceutically acceptable carrier.

14. A pharmaceutical composition comprising at least one dose of an immunotherapeutically effective amount of the monoclonal antibody of claim 1 in combination with at least one additional monoclonal antibody.

15. The pharmaceutical composition according to claim 14 wherein said additional monoclonal antibody is an anti-RSV antibody distinguished from the antibody of claim 1 by virtue of being reactive with a different epitope of the RSV F protein antigen.
